(19) United States
(12) Patent Application Publication
(10) Pub. No.: US 2005/0221328 A1
(43) Pub. Date: Oct. 6, 2005

(54) Inhibitors of inflammatory gene activity and cholesterol biosynthesis

(75) Inventors: Mark J Evans, Radnor, PA (US); Douglas C. Harnish, Pennsburg, PA (US)

Correspondence Address: WILMER CUTLER PICKERING HALE AND DORR LLP, 60 STATE STREET, BOSTON, MA, 02109, US

(73) Assignee: Wyeth

(21) Appl. No.: 10/517,695

(22) PCT Filed: Jun. 13, 2003
(86) PCT No.: PCT/US03/18651

§ 371 Date: Dec. 13, 2004

Related U.S. Application Data
(60) Provisional application No. 60/387,915, filed on Jun. 13, 2002.
(60) Provisional application No. 60/470,188, filed on May 14, 2003.

(51) Int. Cl. C12Q 1/68; C12N 15/85

(52) U.S. Cl. 435/6; 435/455

(57) ABSTRACT

Methods of identifying agents effective as inhibitors of short heterodimer protein (SHP) and farnesoid X receptor (FXR) and promoters, cell lines and vectors used in said methods. Methods of preparing and using the agents effective as inhibitors of short heterodimer protein (SHP), including methods of using same to prevent and/or treat a condition associated with inflammatory gene activity and/or cholesterol biosynthesis in a subject. Agents effective as inhibitors of short heterodimer protein (SHP) and farnesoid X receptor (FXR) and compositions comprising same, including compositions effective in reducing inflammatory gene activity and/or cholesterol biosynthesis in a subject.

### FIELD OF THE INVENTION

The present invention relates to methods of identifying agents effective as inhibitors of inflammatory disease activity and/or cholesterol biosynthesis, and methods of preparing and using compositions comprising the agents to prevent and/or treat conditions that relate to inflammatory disease activity and/or cholesterol biosynthesis, such as atherosclerosis, inflammatory bowel disease, renal disease, etc. The invention relates to agents effective as inhibitors of inflammatory gene activity and/or cholesterol biosynthesis. For example, the invention relates to inhibitors of nuclear receptors, such as short heterodimer protein (SHP) and farnesoid X receptor (FXR). The present invention relates to compositions comprising such inhibitors, including compositions effective at preventing and/or treating diseases or conditions relating to inflammatory gene expression and/or cholesterol biosynthesis. The present invention further relates to infected cell lines and vectors for use in preparing and identifying agents that inhibit inflammatory gene expression and/or cholesterol biosynthesis.

### BACKGROUND OF THE INVENTION

Inflammatory gene expression is associated with an ever increasing number of medical conditions and diseases. For example, atherosclerosis, renal and inflammatory bowel disease have been associated with inflammatory gene expression in the scientific literature. It has been postulated that there likely exists common genetic variants which confer susceptibility to inflammatory diseases or which contribute to the abnormal inflammatory processes shared by subsets of these diseases. The combined impact of these conditions represents a substantial public health issue, particularly where each of these conditions alone represents such a significant medical issue.

The cholesterol biosynthesis pathway is also implicated in significant medical conditions in humans. For example, atherosclerosis, or hardening of the arteries, is the cause of more than half of all mortality in developed countries and the leading cause of death in the United States. When it affects the coronary arteries (e.g., Coronary Heart Disease or CHD), it is the underlying cause of most heart attacks and a common cause of congestive heart failure and arrhythmias.

The pathological process begins very early with a fatty streak composed of lipid deposited in the intima of arteries. Modified macrophages known as foam cells accumulate in the plaque region. These foam cells accumulate lipids, especially oxidized low-density lipoproteins (LDL). These lipoproteins and cholesterol esters induce collagen synthesis by subintimal fibroblasts. When the lesion becomes infiltrated with fibrous material it protrudes into the lumen of the artery. The lesion itself rarely occludes the artery but rather it is blood clots that form on top of the plaque that close off the channel.

Chronic lesions become calcified and the elasticity of the vessel is decreased. This hardening of the arteries causes an increase in resistance to blood flow and therefore an increase in blood pressure. Any vessel in the body may theoretically be affected by atherosclerosis, but the aorta, coronary, carotid and iliac arteries are most frequently affected. Ischemia or infarction of specific regions causes specific symptoms and clinical outcomes.

High blood pressure, elevated cholesterol, low HDL (high density lipoprotein) smoking, diabetes, age, sex, physical inactivity, and family history of heart disease are risk factors for the development of atherosclerosis.

Atherosclerotic disease is a dynamic process and the progression of atheroma formation can be slowed if plasma lipoproteins are reduced. Several pharmacologic approaches are successful dependent on the underlying cause of hyperlipidemia. Dietary measures are implemented first but must be continued as a part of drug therapy.

Most hyperlipidemic patients will respond well to a diet that is restricted in cholesterol and saturated fat. Total fat calories should be 20-25% with saturated fat less than 8% of total calories and cholesterol at less than 200 mg/d. Increased fiber and complex carbohydrates are recommended. Such a diet can reduce serum cholesterol from 20 to 30%.

Niacin (nicotinic acid) decreases plasma LDL levels most probably by inhibiting LDL production. Hepatic cholesterol synthesis is also inhibited. HDL levels are increased due to a decreased break down of HDL. The clotting protein fibrinogen is decreased and the ‘clot buster’ tissue plasminogen activator is increased, both of which may be important in limiting clot formation at plaques.

Side effects include vasodilation at the skin and sensation of warmth, nausea, abdominal discomfort, rashes or dry skin. Even though it is a vitamin it can have some serious side effects at cholesterol lowering dosages, so patients receiving niacin should be monitored by a physician.

Clofibrate increases the clearance of triglyceride-rich lipoproteins and inhibits cholesterol biosynthesis in the liver indirectly. Its action is via the stimulation of enzymes that break down lipoproteins. The most common side effects are abdominal discomfort and nausea. Rare toxic effects include dermatitis, liver dysfunction, bone marrow depression, and sometimes a decreased male libido.

Bile acid binding resins such as colestipol are very large cationic exchange resins that are taken orally. In the gut, they bind bile acids and prevent their reabsorption. The result is an increased excretion of bile acids and lower levels of LDL in plasma. The most common side effects are constipation, bloating, heartburn, and diarrhea.

Neomycin is an antibiotic that inhibits the intestinal resorption of cholesterol and bile acids thereby producing decreases in plasma LDL. Severe side effects may occur with even low doses of neomycin. Nausea, abdominal cramps, diarrhea, and malabsorption may occur. Also, resistant microorganisms may multiply and lead to enterocolitis (inflammation of the intestine and colon).

Statins are currently the most effective cholesterol lowering drugs available. They include atorvastatin, cerivastatin, fluvastatin, lovastatin, pravastatin, and simvastatin. They lower LDL cholesterol (considered the bad cholesterol) and triglycerides in the blood while increasing the HDL cholesterol. HDL cholesterol is considered the “good cholesterol” because it transports cholesterol to the liver where it can be degraded. These drugs mainly differ in their potency, ability to lower triglycerides and cost. However, the statins are generally effective only about 50% of the time or less.

Although cholesterol is well known for its significant adverse effects involving participation in forming the damaging plaques in blood vessels observed in atherosclerosis, cholesterol is also used in the synthesis of steroid hormones and bile acids, and is an important as component of cell membranes. In addition to the cholesterol consumed in the diet, the body also makes cholesterol. It is generally believed that a rate limiting step in the synthesis of cholesterol within cells involves the action of an enzyme called HMG-CoA reductase. The statins, also referred to as the HMG-CoA reductase inhibitors, inhibit this enzyme, decreasing the ability of the cell to synthesize cholesterol.

The damage from atherosclerosis not only results from the atherosclerotic plaque limiting blood flow through narrowed arteries, but also from the rupture of vulnerable plaques. In fact, most of the problems stem from ruptured plaques releasing substances that initiate blood clot formation. Scavenger type cells filled with cholesterol within the plaque secrete substances that make the plaque likely to rupture. The statins appear to stabilize the plaque. Recent studies have also shown that the statins improve the endothelial lining. The endothelium is involved in blood clot formation and lysis, and seems to malfunction in people with coronary disease.

Statins are associated with various side effects and therefore are not well tolerated by many patients. For example, there is a risk of liver toxicity with the statins so blood tests are routinely monitored. Further, there are many drug and some food interactions with these drugs. In some people, the statins cause inflammation of the muscle (myopathy). In fact, this occurs frequently in people receiving some other cholesterol lowering drugs or erythromycin. The occurrence of muscle aches can be very serious. In many cases, the muscle cramps progress to a serious form of muscle inflammation known as rhabdomyolysis and can cause kidney failure. An additional benefit to postmenopausal women of these drugs is that they may reduce the risk of osteoporosis and resultant fractures by stimulating bone growth.

A variety of Over-the-Counter (OTC) supplements that lower cholesterol are made from rice bran oil. They contain a tocotrienol that has activity similar to vitamin E. They act like the statins in decreasing the synthesis of cholesterol by the body. As of yet, their effectiveness and safety has not been established.

Given the deficiencies of the foregoing therapies, a substantial effort has been made to understand the pathways implicated in artherosclerosis. In particular, efforts have been made to understand the cholesterol homeostasis pathway, for example, to identify improved cholesterol reducing agents. For example, SHP has been indicated in the cholesterol homeostasis pathway. Specifically, it has been reported that hepatic bile acid homeostasis is regulated by negative feedback inhibition of genes involved in the uptake and synthesis of bile acids, and that bile acids down-regulate the rate-limiting gene for bile acid synthesis, cholesterol 7alpha-hydroxylase (cyp7a), via bile acid receptor (fxr) activation of SHP, which serves as an inhibitory nuclear receptor. See Denson et al., Gastroenterology, 121(1):218-20 (2001). The protein encoded by the SHP gene is an orphan receptor that contains a putative ligand-binding domain but lacks a conventional DNA-binding domain. The protein is a member of the nuclear hormone receptor family, a group of transcription factors regulated by small hydrophobic hormones, a subset of which do not have known ligands and are referred to as orphan nuclear receptors. The human orphan nuclear hormone receptor SHP protein was initially characterized by Seol, W. et al., Science, 272:1336-39 (1996). However, until now no one has identified compounds capable of inhibiting SHP.

The example of atherosclerosis is illustrative and similar cases may be made for a wide variety of other conditions and diseases, such as renal diseases and inflammatory bowel diseases, for example. In each instance, the previous approaches to preventing and/or treating the condition are deficient in terms of their efficicacy and/or tolerance by patients. Therefore, there remains a significant need for agents that are effective against conditions associated with inflammatory gene expression and/or cholesterol biosynthesis, and that are well tolerated by patients.

Further, there were previously no methods available for efficiently, reliably and cost-effectively identifying agents effective in inhibiting the activity of SHP or FXR in the inflammatory gene pathway and/or cholesterol biosynthesis pathway, or for developing products based thereon that are effective against a condition associated with inflammatory disease expression and/or cholesterol biosynthesis in a subject.

Accordingly, there remains a need to identify agents effective in reducing inflammatory gene expression and/or cholesterol biosynthesis, as well as infected cell lines, vectors and promoters for use in identifying the agents. Further, there remains a need for agents effective as inhibitors of nuclear receptors, such as SHP and FXR, as well as therapeutic compositions comprising same. There also remains a need for compositions effective in reducing elevated cholesterol levels and for preventing and treating atherosclerosis and other conditions associated with elevated cholesterol levels, as well as a need for compositions effective against inflammatory diseases, such as inflammatory bowel diseases and renal diseases. Moreover, there is also a need for compositions and therapies that are generally effective against a broad number of related conditions, such as conditions related to inflammatory gene activity and cholesterol biosynthesis. There is also a need for efficient methods of preparing and administering said compositions.

### SUMMARY OF THE INVENTION

To meet these and other needs, and in view of its purposes, the present invention provides methods for identifying, preparing and administering agents effective as inhibitors of inflammatory disease activity and/or cholesterol biosynthesis, as well as infected cell lines, vectors and promoters for use in said methods. The present methods of identifying the agents are accurate, highly efficient and cost-effective.

An embodiment of the present invention provides a method for identifying an agent effective at inhibiting short heterodimer protein (SHP) or farnesoid X receptor (FXR) comprising: administering an agent to a cell culture that expresses (i) short heterodimer protein (SHP) or (ii) farnesoid X receptor (FXR) and comprises a NF-κB promoter/detectable substance gene reporter; and selecting agents that cause an increase in the detectable substance in the cell culture.

A further embodiment of the present invention provides a method of preventing or ameliorating a condition associated with inflammatory gene activity and/or cholesterol biosynthesis in a subject comprising: administering an agent selected by any of the methods of the invention as described herein.

A further embodiment of the present invention provides a composition comprising an agent selected by any of the methods of the invention as described herein.

A further embodiment of the present invention provides a composition comprising: an agent characterized as causing an increase in luciferase when administered to a cell culture infected, transfected or altered with a vector comprising a nuclear transcription factor NF-κB promoter/luciferase (luc) gene reporter, said cell culture expressing short heterodimer protein (SHP) or farnesoid X receptor (FXR).

A further embodiment of the present invention provides a promoter/detectable substance gene reporter comprising: an NF-κB promoter and a detectable substance gene, said NF-κB promoter positioned in front of the detectable substance gene.

A further embodiment of the present invention provides an isolated CYP7A1, CYP8B1 or SHP promoter comprising: a polynucleotide comprising the nucleic acid sequence of SEQ ID NO: 5, SEQ ID NO:6, SEQ ID NO:7, SEQ ID NO:8 or a combination thereof.

A further embodiment of the present invention provides a composition comprising: a non-naturally deactivated short heterodimer protein (SHP) or farnesoid X receptor (FXR) complex.

A further embodiment of the present invention provides a composition comprising: an agent that binds to any of SEQ ID NOS:1-4.

It is to be understood that the foregoing general description and the following detailed description are exemplary, but are not restrictive, of the invention.

### BRIEF DESCRIPTION OF THE DRAWINGS

FIG. 1 is a line graph that illustrates the inhibition of IL-1β gene expression by ethynylestradiol.

FIG. 2 is a line graph that that illustrates the inhibition of SHP expression in HepG2 by IL-1β.

FIGS. 3a, 3b and 3c are bar graphs which illustrate that ethynylestradiol inhibition of Fnk, JAB and LIX is specific for IL-1β.

FIGS. 4a, 4b, 4c, 4d, and 4e are bar graphs which illustrate that ethynylestradiol blocks IL-1β induction of gene expression by an estrogen receptor (ER) dependent mechanism.

FIGS. 5a, 5b, 5c, 5d, and 5e are bar graphs which illustrate that ERα is required for ethynylestradiol regulation in the liver.

FIGS. 6a and 6b are bar graphs that illustrate the ethynylestradiol induction of gene expression is not required for ERα inhibition of IL-1β gene inductions.

FIGS. 7a, 7b, 7c, 7d, and 7e are bar graphs that illustrate that ethynylestradiol inhibition of IL-1β is absent in the lung.

FIG. 8 is a bar graph depicting ERα and Erβ inhibition of IL-1β induction of NFκB activity in HepG2 cells.

FIG. 9 is a bar graph depicting the regulation of SHP expression in mouse liver by ERα.

FIG. 10 is a bar graph that illustrates the regulation of SHP by estrogen in the rat liver.

FIGS. 11a and 11b are bar graphs which illustrate that ERα regulates hSHP promoter activity in human cells.

FIGS. 12a and 12b are bar graphs illustrating that ERα regulates hSHP promoter activity in 293 cells.

FIG. 12c is a line graph illustrating that ERα regulates hSHP promoter activity in 293 cells.

FIG. 13 is a map illustrating the location of the E2 response element in 293 cells.

FIGS. 14a, 14b, and 14c are bar graphs which illustrate that ER induction of SHP fails to repress CYP7A1 and CYP8B1.

FIG. 15 is a line graph which illustrates that cholate repression of CYP7A1 and CYP8B1 does not require SHP induction.

FIG. 16 depicts luciferase activity resulting from tittering SHP/pSI against a CYP8B1 promoter driven by HNF4/pSI in HepG2.

FIG. 17 is a graph illustrating dose dependent induction of the hepatic levels of TNF-alpha, VCAM-1 and RANTES mRNA.

FIGS. 18a and 11b are graphs illustrating relative SHP expression with UDCA and CA

FIG. 19 is a graph illustrating relative expression of CDCA in HepG2 cells.

FIG. 20a is a graph illustrating relative expression of GW 4064 in HepG2 cells.

FIG. 20b is a graph illustrating M-CSF expression induced in a dose dependent manner by CDCA or GW 4064 treatment.

FIGS. 21a-d are bar graphs illustrating inflammatory gene expression dependence on cholate.

FIGS. 22a-b are line graphs illustrating that acute cholate treatment induces inflammatory gene expression in a dose dependent fashion.

FIGS. 23a-b are bar graphs illustrating that UDCA does not induce inflammatory gene expression.

FIGS. 24a-b are line graphs illustrating that FXR agonists induce ICAM-1 expression in HepG2 cells.

FIG. 25 is a line graph illustrating that GW 4064 induces ICAM-1 expression in vivo.

FIG. 26 is a bar graph illustrating that GW 4064 induces ICAM-1 promoter expression.

### DETAILED DESCRIPTION OF THE INVENTION

Bile acids are amphiphatic steroid detergents necessary for the digestion and absorption of fat-soluble nutrients from the intestine and have been shown to be ligands for the nuclear receptor farnesoid X receptor (FXR) that regulates bile acid and cholesterol metabolism. Bile acids are synthesized in the liver, excreted into the bile, reabsorbed in the ileum, and transported back to the liver via portal circulation to inhibit bile acid synthesis by suppressing the gene encoding the rate-limiting enzyme, cholesterol 7α-hydroxylase (cyp7a). FXR-mediated induction of the orphan nuclear receptor SHP (short heterodimer partner) results in repression of cyp7a through SHP's antagonism of LRH-1 (liver receptor homolog-1) activity, a transcription factor that controls cyp7a expression (Goodwin '00 Cell & Lu '00 Cell). Therefore, the therapeutic potential of FXR and SHP antagonists in controlling hyperlipidemia and cholestatic liver disease has been proposed.

It has been unexpectedly discovered that a novel function for FXR signaling pathways relate to the potential to induce inflammatory gene expression in the liver. Bile acids have previously been demonstrated to induce inflammatory cytokines in hepatic macrophages (Miyake et al '00 JBC 275:21805) through an uncharacterized mechanism. Besides signaling though FXR, bile acids have been shown to directly activate protein kinase C signaling pathways (Stravitz '95 JLR) that could also result in inflammatory gene expression. Here we demonstrate the ability of activated FXR to directly activate inflammatory gene expression in the hepatocyte cell line, HepG2. Treatment of HepG2 cells with the bile acid, chenodeoxycholic acid (CDCA), or the synthetic FXR ligand GW 4064 resulted in induction of the inflammatory genes intracellular adhesion molecule-(ICAM-1) and macrophage-colony stimulating factor (M-CSF). Further the following evidence indicates that FXR signaling induces NF-κB mediated inflammatory gene expression through the induction of SHP expression. These results suggest a new role for activated FXR in promoting inflammatory gene expression. Since FXR expression is predominantly in the liver, intestine, adrenal and kidney an FXR or SHP antagonist could demonstrate anti-inflammatory activity in diseases such as atherosclerosis, inflammatory bowel diseases and in renal diseases, for example.

Based upon the foregoing unexpected discoveries, methods for identifying agents that inhibit FXR and/or SHP in the cholesterol biosynthesis pathway and/or the inflammatory gene expression pathway are provided herein.

The terms “composition” or “therapeutic composition” and “compositions” or “therapeutic compositions”, respectively, are used interchangeably herein. Thus, the plural includes the singular and the singular includes the plural form of the respective terms.

Description of the compositions as “comprising” certain ingredients also includes those compositions which consist essentially of or consist of the same ingredients.

As used herein the term “therapeutic composition” means a composition useful for treating cells, tissues, organs or systems, both internally and externally.

As used herein the term “therapeutically effective amount” means an amount effective to treat the target medical condition.

The term “pharmaceutically-acceptable”, as used herein, means that the compositions or components thereof so described are of sufficiently high purity and suitable for use in contact with skin, tissues, or membranes without undue toxicity, incompatibility, instability, allergic response, and the like.

The term “elevated cholesterol levels” refers to serum levels of low-density lipoprotein (LDL) cholesterol that would be considered by persons of ordinary skill in the art to be unhealthy and an appropriate target for treatment of some sort. Currently, a serum LDL cholesterol level greater than 220 mg per dL (5.7 mmol per L) is considered unhealthy and is targeted for treatment.

The invention relates to methods of preparing and identifying agents and compositions effective as inhibitors of inflammatory disease activity and/or cholesterol biosynthesis and methods of using the compositions to prevent and/or treat inflammatory disease or conditions associated with cholesterol biosynthesis in a subject. The present invention further relates to infected cell lines and vectors for use in preparing and identifying agents that inhibit inflammatory gene expression and/or cholesterol biosynthesis.

The present invention also provides agents identified by the methods of the present invention that are effective as inhibitors of inflammatory gene activity and/or cholesterol biosynthesis. For example, the invention relates to inhibitors of nuclear receptors, such as short heterodimer protein (SHP) and farnesoid X receptor (FXR), and particularly to inhibitors of SHP and FXR in the inflammatory gene pathway and/or cholesterol biosynthesis pathway. The present invention relates to compositions comprising such inhibitory agents, including compositions effective at preventing and/or treating diseases or conditions relating to inflammatory gene expression and/or cholesterol biosynthesis, such as atherosclerosis, inflammatory bowel disease and renal disease.

Further, the present invention provides compositions and methods to ameliorate and prevent elevated cholesterol levels and related illnesses, such as atherosclerosis. To identify compositions useful for the amelioration and prevention of elevated cholesterol levels and associated conditions, a method is provided herein that is highly efficient and effective in screening for agents that inhibit SHP activity in the cholesterol homeostasis pathway.

The therapeutic compositions of the present invention can be used alone or together with any other medical and/or treatment approach. No adverse side effects or problems associated with the use of the therapeutic compositions of the invention are known.

The compositions and methods of the present invention are appropriate for individuals without a conditions, but who may be deemed to be at a higher than normal risk for a certain condition or combination of conditions.

For example, the compositions and methods of the invention are also appropriate in some cases for individuals without elevated cholesterol levels, but who are determined to be at risk for elevated levels of cholesterol or illnesses related thereto. A wide variety of risk factors, such as high blood pressure, elevated cholesterol, low HDL (high density lipoprotein) smoking, diabetes, age, sex (white male death rate at ages 35 to 44 is 6 times that of white females), physical inactivity, family history of heart disease, etc. may be taken into consideration as would be known to persons of ordinary skill in the art. For example, without limitation, the compositions of the invention may be administered to persons who are found to be genetically predisposed to elevated cholesterol levels or illness generally related thereto, such as atherosclerosis, persons having low levels of low-density lipoprotein (LDL), persons with high blood pressure, persons at certain life stages, etc. In the case of low LDL for example, while elevated serum high-density lipoprotein (HDL) cholesterol levels are thought to be cardioprotective, a low level is a potent predictor of premature CHD (Coronary Heart Disease). An isolated low HDL level is defined as an HDL cholesterol level of 35 mg per dL (0.90 mmol per L) or less, an LDL cholesterol level of less than 160 mg per dL (4.15 mmol per L) and a triglyceride level of less than 250 mg per dL (2.83 mmol per L). Of course, any of these risk factors could change over time as new research becomes available. The present invention is intended to contemplate any such changes. Further, persons of ordinary skill in the art would readily be able to identify such situations and administer the compositions of the invention appropriately, based upon the guidance provided herein.

Nuclear hormone receptors are ligand-activated transcription factors that are involved in a variety of physiological, developmental, and toxicological processes (Mangelsdorf et al., 1995; Blumberg and Evans, 1998; Kliewer et al., 1999). These receptors have highly conserved DNA and ligand binding domains and are sub-classified into two groups based upon their dimerization properties. The first group includes the estrogen, progesterone, and glucocorticoid receptors, all of which bind as homodimers to their cognate DNA response elements. The second group includes the peroxisome proliferator-activated receptor (PPAR), retinoic acid receptor (RAR), vitamin D receptor, and thyroid hormone receptor, all of which form heterodimers with a common partner known as the retinoid X receptor (RXR).

The farnesoid X receptor (FXR), which was isolated from a rat liver cDNA library using a degenerate oligonucleotide probe derived from the highly conserved nuclear receptor superfamily DNA binding domain (Forman et al., 1995), belongs to this second group. High concentrations of farnesol, an isoprene metabolite of the mevalonate pathway, were found to activate FXR (Forman et al., 1995). The mouse ortholog RIP14 was cloned using the human RXR ligand binding domain as bait in the yeast two-hybrid assay and was found to be activated only poorly by farnesoids (Seol et al., 1995) Instead, retinoic acid and the synthetic retinoid TTNPB {(E)-4-[2-(5,6,7,8-tetrahydro-5,5,8,8-tetramethyl-2-naphthylenyl)-1propenyl] benzoic acid} were demonstrated to be the potent activators of FXR, albeit at supraphysiological concentrations (Zaviacki et al., 1997). While farnesoids and retinoid allow valuable initial characterizations of FXR, the high concentrations required for activation suggest that these compounds are precursors to endogenous ligands or are mimicking the actions of some other relevant physiological ligand(s).

SHP is a member of the nuclear hormone receptor superfamily with no known ligand. Molecular modeling by homology of the SHP protein suggests that it can achieve the classical ligand bound receptor conformation, as shown below in the figure in which SHP (white strand) is modeled on the ligand binding domain of RXRs complexed with gaie-retinoic acid (red strand). SHP contains a classical helix 12 including the necessary hydrophobic amino acids and negatively charged amino acid necessary for the formation of a charge clamp interaction with coactivator or corepressors.

The following is the nucleic acid sequence for the gene that encodes short heterodimer protein (SHP), designated herein as SEQ ID NO:1.

```
NUCLEIC ACID SEQUENCE OF SHP GENE
(SEQ ID NO:1)
      1 gagctggaag tgagagcaga tccctaacca tgagcaccag ccaaccaggg gcctgcccat
    61 gccagggagc tgcaagccgc cccgccattc tctacgcact tctgagctcc agcctcaagg
  121 ctgtcccccg accccgtagc cgctgcctat gtaggcagca ccggcccgtc cagctatgtg
  181 cacctcatcg cacctgccgg gaggccttgg atgttctggc caagacagtg gccttcctca
  241 ggaacctgcc atccttctgg cagctgcctc cccaggacca gcggcggctg ctgcagggtt
  301 gctggggccc cctcttcctg cttgggttgg cccaagatgc tgtgaccttt gaggtggctg
  361 aggccccggt gcccagcata ctcaagaaga ttctgctgga ggagcccagc agcagtggag
  421 gcagtggcca actgccagac agaccccagc cctccctggc tgcggtgcag tggcttcaat
  481 gctgtctgga gtccttctgg agcctggagc ttagccccaa ggaatatgcc tgcctgaaag
  541 ggaccatcct cttcaacccc gatgtgccag gcctccaagc cgcctcccac attgggcacc
  601 tgcagcagga ggctcactgg gtgctgtgtg aagtcctgga accctggtgc ccagcagccc
  661 aaggccgcct gacccgtgtc ctcctcacgg cctccaccct caagtccatt ccgaccagcc
  721 tgcttgggga cctcttcttt cgccctatca ttggagatgt tgacatcgct ggccttcttg
  781 gggacatgct tttgctcagg tgacctgttc cagcccaggc agagatcagg tgggcagagg
  841 ctggcagtgc tgattcagcc tggccatccc cagaggtgac ccaatgctcc tggaggggca
  901 agcctgtata gacagcactt ggctccttag gaacagctct tcactcagcc acaccccaca
  961 ttggacttcc ttggtttgga cacagtgctc cagctgcctg ggaggctttt ggtggtcccc
1021 acagcctctg ggccaagact cctgtccctt cttgggatga gaatgaaagc ttaggctgct
1081 tattggacca gaagtcctat cgactttata cagaactgaa ttaagttatt gatttttgta
1141 ataaaaggta tgaaacacta aaaaaaaa
```

The following is the amino acid sequence of the short heterodimer protein (SHP) polypeptide, which is designated herein as SEQ ID NO:2.

```
AMINO ACID SEQUENCE OF SHP POLYPEPTIDE
(SEQ ID NO:2)
    1 MSTSQPGACP CQGAASRPAI LYALLSSSLK AVPRPRSRCL CRQHRPVQLC APHRTCREAL
  61 DVLAKTVAFL RNLPSFWQLP PQDQRRLLQG CWGPLFLLGL AQDAVTFEVA EAPVPSILKK
121 ILLEEPSSSG GSGQLPDRPQ PSLAAVQWLQ CCLESFWSLE LSPKEYACLK GTILFNPDVP
181 GLQAASHIGH LQQEAHWVLC EVLEWCPAA QGRLTRVLLT ASTLKSIPTS LLGDLFFRPI
241 IGDVDIAGLL GDMLLLR
```

The following is the nucleic acid sequence of the gene the encodes FXR:

NUCLEIC ACID SEQUENCE OF FXR GENE
(SEQ ID NO:3)

```
      1 acgagactct ctcctcCtCc tcacctcatt gtctccccga cttatcctaa tgcgaaattg
    61 gattctgagc atttgtagca aaatcgctgg gatctggaga ggaagactca gtccagaatc
  121 ctcccagggc cttgaaagtc catctctgac ccaaaacaat ccaaggaggt agaagacatc
  181 gtagaaggag tgaaagaaga aaagaagact tagaaacata gctcaaagtg aacactgctt
  241 ctcttagttt cctggatttc ttctggacat ttcctcaaga tgaaacttca gacactttgg
  301 agtttttttt gaagaccacc ataaagaaag tgcatttcaa ttgaaaaatt tggatgggat
  361 caaaaatgaa tctcattgaa cattcccatt tacctaccac agatgaattt tctttttctg
  421 aaaatttatt tggtgtttta acagaacaag tggcaggtcc tctgggacag aacctggaag
  481 tggaaccata ctcgcaatac agcaatgttc agtttcccca agttcaacca cagatttcct
  541 cgtcatccta ttattccaac ctgggtttct acccccagca gcctgaagag tggtactctc
  601 ctggaatata tgaactcagg cgtatgccag ctgagactct ctaccaggga gaaactgagg
  661 tagcagagat gcctgtaaca aagaagcccc gcatgggcgc gtcagcaggg aggatcaaag
  721 gggatgagct gtgtgttgtt tgtggagaca gagcctctgg ataccactat aatgcactga
  781 cctgtgaggg gtgtaaaggt ttcttcagga gaagcattac caaaaacgct gtgtacaagt
  841 gtaaaaacgg gggcaactgt gtgatggata tgtacatgcg aagaaagtgt caagagtgtc
  901 gactaaggaa atgcaaagag atgggaatgt tggctgaatg cttgttaact gaaattcagt
  961 gtaaatctaa gcgactgaga aaaaatgtga agcagcatgc agatcagacc gtgaatgaag
1021 acagtgaagg tcgtgacttg cgacaagtga cctcgacaac aaagtcatgc agggagaaaa
1081 ctgaactcac cccagatcaa cagactcttc tacattttat tatggattca tataacaaac
1141 agaggatgcc tcaggaaata acaaataaaa ttttaaaaga agaattcagt gcagaagaaa
1201 attttctcat tttgacggaa atggcaacca atcatgtaca ggttcttgta gaattcacaa
1261 aaaagctacc aggatttcag actttggacc atgaagacca gattgctttg ctgaaagggt
1321 ctgcggttga agctatgttc cttcgttcag ctgagatttt caataagaaa cttccgtctg
1381 ggcattctga cctattggaa gaaagaattc gaaatagtgg tatctctgat gaatatataa
1441 cacctatgtt tagtttttat aaaagtattg gggaactgaa aatgactcaa gaggagtatg
1501 ctctgcttac agcaattgtt atcctgtctc cagatagaca atacataaag gatagagagg
1561 cagtagagaa gcttcaggag ccacttcttg atgtgctaca aaagttgtgt aagattcacc
1621 agcctgaaaa tcctcaacac tttgcctgtc tcctgggtcg cctgactgaa ttacggacat
1681 tcaatcatca ccacgctgag atgctgatgt catggagagt aaacgaccac aagtttaccc
1741 cacttctctg tgaaatctgg gacgtgcagt gatggggatt acaggggagg ggtctagctc
1801 ctttttctct ctcatattaa tctgatgtat aactttcctt tatttcactt gtacccagtt
1861 tcactcaaga aatcttgatg aatatttatg ttgtaattac atgtgtaact tccacaactg
1921 taaatattgg gctagataga acaactttct ctacattgtg ttttaaaagg ctccagggaa
1981 tcctgcattc taattggcaa gccctgtttg cctaattaaa ttgattgtta cttcaattct
2041 atctgttgaa ctagggaaaa tctcattttg ctcatcttac catattgcat atattttatt
2101 aaagagttgt attcaatctt ggcaataaag caaacataat ggcaacagaa aaaaaaaaaa
2161 aaaaaaaaaa aaaaaaaaaa aaaaaaaaaa aaaaaaaaaa aaaaaaaaaa aaaaaaaa
```

The following is the amino acid sequence of FXR.

```
AMINO ACID SEQUENCE OF FXR POLYPEPTIDE
(SEQ ID NO:4)
    1 MGSKMNLIEH SHLPTTDEFS FSENLFSVLT EQVAGPLGQN LEVEPYSQYS NVQFPQVQPQ
  61 ISSSSYYSNL GFYPQQPEEW YSPGIYELRR MPAETLYQGE TEVAEMPVTK KPRMGASAGR
121 IKGDELCVVC GDRASGYHYN ALTCEGCKGF FRRSITKNAV YKCKNGGNCV MDMYMRRKCQ
181 ECRLRKCKEM GMLAECLLTE IQCKSKRLRK NVKQHADQTV NEDSEGRDRL QVTSTTKSCR
241 EKTELTPDQQ TLLHFIMDSY NKQRMPQEIT NKILKEEFSA EENFLILTEM ATNHVQVLVE
301 FTKKLPGFQT LDHEDQIALL KGSAVEAMFL RSAEIFNKKL PSGHSDLLEE RIRNSGISDE
361 YITPMFSFYK SIGELKMTQE EYALLTAIVI LSPDRQYIKD REAVEKLQEP LLDVLQKLCK
421 IHQPENPQHF ACLLGRLTEL RTFNHHHAEM LMSWRVNDHK FTPLLCEIWD VQ
```

The present invention relates to the identification and/or development of agents (e.g., small molecules, antisense oligonucleotides, recombinant receptors, antibodies, etc.) that inhibit the activity of SHP and/or FXR in the inflammatory gene pathway and/or the cholesterol biosynthesis pathway, for example by binding to or competing with SHP or FXR. These agents are able to inhibit SHP's or FXR's negative effects on bile acid synthesis in the cholesterol homeostasis pathway and thereby effectively lower serum LDL cholesterol levels and/or inhibit SHP's or FXR's negative effects in the inflammatory gene expression pathway.

Methods of Identifying SHP or FXR Inhibitors

Because there is no known ligand for SHP or FXR, binding assays to identify molecules effecting their activity are not possible. The present invention is directed to a novel approach for identifying agents that inhibit SHP and/or FXR activity in the inflammatory gene expression and/or the cholesterol biosynthesis pathways.

As the examples herein demonstrate, we have established that estrogen can induce expression of SHP through an ERα dependent mechanism. This induction of SHP would be predicated to decrease bile acid synthesis and bile acids levels in the hepatocyte. This could result in diminished activity of FXR and diminished production of apoCII with accompanying increases in plasma triglycerides. Further, a decrease in bile acid synthesis could lead to an increased propensity for gall stone formation due to decreased ratio of bile acid to cholesterol in the bile. Interestingly, women on hormone replacement therapy exhibit these two effects: they have increased triglyceride levels and increased incidence of gall stones. This suggests that pharmacological manipulation of SHP activity results in observable physiological changes. Inhibition of SHP activity thus increases conversion of cholesterol to bile acids and decrease triglyceride levels. These are very favorable effects in patients with atherosclerosis.

According to an embodiment of the present invention, a method of identifying a SHP or FXR inhibitor in the inflammatory gene expression pathway and/or cholesterol biosynthesis pathway comprises a screening assay. The screening assay involves obtaining a candidate agent and then administered the candidate agent to a cell culture that expresses (i) SHP or (ii) FXR and comprises a NF-κB promoter/detectable substance gene reporter. Candidate agents that inhibit SHP and/or FXR will cause an increase in the detectable substance. Thus, effective inhibitors may be selected from any of a number of possible candidate agents by monitoring for an increase in the detectable substance in the infected cell culture after administration of each candidate.

In accordance with an embodiment, the NF-κB promoter comprises a Thymidine Kinase (TK) promoter and then upstream of the TK promoter there are two copies of the NF-κB response element.

Another method of identifying SHP inhibitors according to an embodiment of the present invention, generally involves constructing full length LRH-1 and SHP clones, building LRH-1 reporters, developing adenovirus constructs, selecting cell lines and establishing a screening paradigm. According to an implementation of the present invention, the process involves: cloning a CYP7A1 or CYP8B1 promoter; inserting the cloned CYP7A1 or CYP8B1 promoter into a vector ahead of a detectable substance gene (e.g., luciferase (luc) gene) to form a CYP7A1 or CYP8B1 promoter/detectable substance gene (e.g. luciferase (luc) gene) reporter; infecting, transfecting or altering (e.g, chromosomal change, etc.) a cell culture that expresses SHP, and optionally HNF4α, with the CYP7A1 or CYP8B1 promoter/detectable substance gene (e.g., luciferase (luc) gene) reporter to form an infected cell culture; administering the agent to the infected cell culture; and detecting an increase in the detectable substance (e.g., luciferase) in the infected cell culture.

Methods for identifying certain agents that are effective as inhibitors in both the cholesterol biosynthesis pathway and/or the inflammatory gene expression pathway are provided herein. According to another implementation of the present invention, the process involves: cloning a CYP7A1 or CYP8B1 promoter; inserting the cloned CYP7A1 or CYP8B1 promoter into a vector ahead of a detectable substance gene (e.g., luciferase (luc) gene) to form a CYP7A1 or CYP8B1 promoter/detectable substance gene (e.g. luciferase (luc) gene) reporter; infecting a cell culture that expresses SHP and optionally HNF4a with the CYP8B1 promoter/detectable substance gene (e.g., luciferase (luc) gene) reporter to form an infected cell culture; administering the agent to the infected cell culture; and detecting an increase in the detectable substance (e.g., luciferase) in the infected cell culture; cloning a CYP7A1 or CYP8B1 promoter; inserting the cloned CYP7A1 or CYP8B1 promoter into a vector ahead of a detectable substance gene (e.g., luciferase (luc) gene) to form a CYP7A1 or CYP8B1 promoter/detectable substance gene (e.g. luciferase (luc) gene) reporter; infecting a cell culture that expresses SHP, and optionally HNF4α, with the CYP7 μl or CYP8B1 promoter/detectable substance gene (e.g., luciferase (luc) gene) reporter to form an infected cell culture; administering the agent to the infected cell culture; and detecting an increase in the detectable substance (e.g., luciferase) in the infected cell culture.

In accordance with an embodiment, protein levels of SHP and/or FXR are monitored to determine whether the agent is acting on the protein or perhaps at the nucleotide level. For example, cells expressing SHP may be tagged (e.g., by epitope tagging or using a flag tag of six or seven amino acids) and then levels monitored by using Western Blot, ELISA or some suitable technique, as would be known to persons skilled in the art.

The following disclosure describes various embodiments for practicing the above described method. Further, specific examples are provided below (see Examples 3 and 4) that show the complete performance of specific assays to identify any candidate compound as a SHP inhibitor. Example 3 provides a specific procedure for performing the assay according to one implementation of the invention using transfection of a cell culture with a plasmid. Example 4 provides the preferred specific procedure for infecting the cell culture with an adenovirus. Using the method of the present invention, SHP and FXR inhibitors may be identified. Based upon the disclosure provided herein, a person of ordinary skill in the art would readily be able to practice said method in accordance with the various embodiments of the invention. Therefore, persons of ordinary skill in the art would be readily able to structure assays for identifying SHP or FXR inhibitors based upon the information provided herein.

Expression Systems and Vectors

Host cells (and cell cultures comprising same) are genetically engineered to incorporate expression systems, portions thereof, or polynucleotides of the invention. Introduction of polynucleotides into host cells are effected, for example, by methods described in many standard laboratory manuals, such as Davis et al., Basic Methods in Molecular Biology (1986) and Sambrook et al., Molecular Cloning: A Laboratory Manual, 2nd ed., Cold Spring Harbor Laboratory Press, Cold Spring Harbor, N.Y. (1989), such as calcium phosphate transfection, DEAE-dextran mediated transfection, transvection, microinjection, ultrasound, cationic lipid-mediated transfection, electroporation, transduction, scrape loading, ballistic introduction, or infection.

Representative examples of suitable hosts include mammalian cells, such as rat cells (e.g., cultured rat hepatoma cells), mouse cells (e.g., mouse hepatocyte cells), rabbit cells, human cells and the like. For example, suitable cells include HELA, human hepatoblastoma cell line (HepG2), human embryonic kidney 293 cell line (HEK293), rat FTO-2B cells, rat McA-RH7777 or combinations thereof.

The recombinantly produced polypeptides are recovered and purified from recombinant cell cultures by well-known methods, including high performance liquid chromatography, ammonium sulfate or ethanol precipitation, acid extraction, anion or cation exchange chromatography, phosphocellulose chromatography, hydrophobic interaction chromatography, affinity chromatography, hydroxylapatite chromatography, and lectin chromatography.

A great variety of expression systems are used. Such systems include, among others, chromosomal, episomal and virus-derived systems, e.g., vectors derived from bacterial plasmids, attenuated bacteria such as Salmonella (U.S. Pat. No. 4,837,151) from bacteriophage, from transposons, from yeast episomes, from insertion elements, from yeast chromosomal elements, from viruses such as vaccinia and other poxviruses, sindbis, adenovirus, baculoviruses, papova viruses, such as SV40, fowl pox viruses, pseudorabies viruses and retroviruses, alphaviruses such as Venezuelan equine encephalitis virus (U.S. Pat. No. 5,643,576), nonsegmented negative-stranded RNA viruses such as vesicular stomatitis virus (U.S. Pat. No. 6,168,943), and vectors derived from combinations thereof, such as those derived from plasmid and bacteriophage genetic elements, such as cosmids and phagemids. The expression systems should include control regions that regulate as well as engender expression, such as promoters and other regulatory elements (such as a polyadenylation signal). Generally, any system or vector suitable to maintain, propagate or express polynucleotides to produce a polypeptide in a host may be used. The appropriate nucleotide sequence may be inserted into an expression system by any of a variety of well-known and routine techniques, such as, for example, those set forth in Sambrook et al., Molecular Cloning, A Laboratory Manual (supra); see also Zhang et al., Journal of Biological Chemistry, 276(45):41690-41699 (2001); Goodwin et al., Molecular Cell, 6:517-526 (2000); and Sinal et al. Cell, 102:731-744 (2000), each of which is incorporated herein in its entirety.

The invention also provides vectors (e.g., expression vectors, sequencing vectors, cloning vectors) which comprise a polynucleotide or polynucleotides of the invention, host cells which are genetically engineered with vectors of the invention, and production of polypeptides of the invention by recombinant techniques. Cell-free translation systems can also be employed to produce such proteins using RNAs derived from the DNA constructs of the invention.

Preferred vectors are viral vectors, such as lentiviruses, retroviruses, herpes viruses, adenoviruses, adeno-associated viruses, vaccinia virus, baculovirus, and other recombinant viruses with desirable cellular tropism. Thus, a gene encoding a functional or mutant protein or polypeptide, or fragment thereof can be introduced in vitro using a viral vector or through direct introduction of DNA. Expression in targeted tissues can be effected by targeting the transgenic vector to specific cells, such as with a viral vector or a receptor ligand, or by using a tissue-specific promoter, or both. Targeted gene delivery is described in PCT Publication Number WO 95/28494.

Viral vectors commonly used for in vitro procedures are DNA-based vectors and retroviral vectors. Methods for constructing and using viral vectors are known in the art (e.g., Miller and Rosman, BioTechniques, 1992, 7:980-990). Preferably, the viral vectors are replication-defective, that is, they are unable to replicate autonomously in the target cell. Preferably, the replication defective virus is a minimal virus, i.e., it retains only the sequences of its genome which are necessary for encapsulating the genome to produce viral particles.

DNA viral vectors include an attenuated or defective DNA virus, such as, but not limited to, herpes simplex virus (HSV), papillomavirus, Epstein Barr virus (EBV), adenovirus, adeno-associated virus (AAV), and the like. Defective viruses, which entirely or almost entirely lack viral genes, are preferred. A defective virus is not infective after introduction into a cell. Use of defective viral vectors allows for administration to cells in a specific, localized area, without concern that the vector can infect other cells. Thus, a specific tissue can be specifically targeted. Examples of particular vectors include, but are not limited to, a defective herpes virus 1 (HSV 1) vector (Kaplitt et al., Molec. Cell. Neurosci., 1991, 2:320-330), defective herpes virus vector lacking a glycoprotein L gene, or other defective herpes virus vectors (PCT Publication Numbers WO 94/21807 and WO 92/05263); an attenuated adenovirus vector, such as the vector described by Stratford-Perricaudet et al. (J. Clin. Invest., 1992, 90:626-630; see also La Salle et al., Science, 1993, 259:988-990); and a defective adeno-associated virus vector (Samulski et al., J. Virol., 1987, 61:3096-3101; Samulski et al., J. Virol., 1989, 63:3822-3828; Lebkowski et al., Mol. Cell. Biol., 1988, 8:3988-3996).

Various companies produce viral vectors commercially, including, but not limited to, Avigen, Inc. (Alameda, Calif.; AAV vectors), Cell Genesys (Foster City, Calif.; retroviral, adenoviral, AAV vectors, and lentiviral vectors), Clontech (retroviral and baculoviral vectors), Genovo, Inc. (Sharon Hill, Pa.; adenoviral and AAV vectors), Genvec (adenoviral vectors), IntroGene (Leiden, Netherlands; adenoviral vectors), Molecular Medicine (retroviral, adenoviral, AAV, and herpes viral vectors), Norgen (adenoviral vectors), Oxford BioMedica (Oxford, United Kingdom; lentiviral vectors), and Transgene (Strasbourg, France; adenoviral, vaccinia, retroviral, and lentiviral vectors).

Adenoviruses are eukaryotic DNA viruses that can be modified to efficiently deliver a nucleotide of the invention to a variety of cell types. Various serotypes of adenovirus exist. Of these serotypes, preference is given, within the scope of the invention, to using type 2 or type 5 human adenoviruses (Ad 2 or Ad 5) or adenoviruses of animal origin (See, PCT Publication Number WO 94/26914.). Those adenoviruses of animal origin which can be used within the scope of the invention include adenoviruses of canine, bovine, murine (e.g., Mav1, Beard et al., Virology, 1990, 75-81), ovine, porcine, avian, and simian (e.g., SAV) origin. Preferably, the adenovirus of animal origin is a canine adenovirus, more preferably a CAV2 adenovirus (e.g., Manhattan or A26/61 strain, ATCC VR-800, for example). Various replication defective adenovirus and minimum adenovirus vectors have been described (e.g., PCT Publication Numbers WO 94/26914, WO 95/02697, WO 94/28938, WO 94/28152, WO 94/12649, WO 95/02697, WO 96/22378). The replication defective recombinant adenoviruses according to the invention can be prepared by any technique known to the person skilled in the art (e.g., Levrero et al., Gene, 1991, 101:195; European Publication Number EP 185 573; Graham, EMBO J., 1984, 3:2917; Graham et al., J. Gen. Virol., 1977, 36:59). Recombinant adenoviruses are recovered and purified using standard molecular biological techniques, which are well known to one of ordinary skill in the art.

The adeno-associated viruses (AAV) are DNA viruses of relatively small size that can integrate, in a stable and site-specific manner, into the genome of the cells which they infect. They are able to infect a wide spectrum of cells without inducing any effects on cellular growth, morphology, or differentiation, and they do not appear to be involved in human pathologies. The AAV genome has been cloned, sequenced, and characterized. The use of vectors derived from the AAVs for transferring genes in vitro has been described (See, PCT Publication Numbers WO 91/18088 and WO 93/09239; U.S. Pat. Nos. 4,797,368 and 5,139,941; European Publication Number EP 488 528). The replication defective recombinant AAVs according to the invention can be prepared by cotransfecting a plasmid containing the nucleic acid sequence of interest flanked by two AAV inverted terminal repeat (ITR) regions, and a plasmid carrying the AAV encapsidation genes (rep and cap genes), into a cell line which is infected with a human helper virus (for example, an adenovirus). The AAV recombinants which are produced are then purified by standard techniques.

In another embodiment, the gene can be introduced in a retroviral vector, e.g., as described in U.S. Pat. No. 5,399,346; Mann et al., Cell, 1983, 33:153; U.S. Pat. Nos. 4,650,764 and 4,980,289; Markowitz et al., J. Virol., 1988, 62:1120; U.S. Pat. No. 5,124,263; European Publication Numbers EP 453 242 and EP178 220; Bernstein et al., Genet. Eng., 1985, 7:235; McCormick, BioTechnology, 1985, 3:689; PCT Publication Number WO 95/07358; and Kuo et al., Blood, 1993, 82:845. The retroviruses are integrating viruses that infect dividing cells. The retrovirus genome includes two LTRs, an encapsidation sequence, and three coding regions (gag, pol and env). In recombinant retroviral vectors, the gag, pol and env genes are generally deleted, in whole or in part, and replaced with a heterologous nucleic acid sequence of interest. These vectors can be constructed from different types of retrovirus, such as, HIV, MoMuLV (“murine Moloney leukaemia virus”), MSV (“murine Moloney sarcoma virus”), HaSV (“Harvey sarcoma virus”), SNV (“spleen necrosis virus”), RSV (“Rous sarcoma virus”), and Friend virus. Suitable packaging cell lines have been described, in particular the cell line PA317 (U.S. Pat. No. 4,861,719), the PsiCRIP cell line (PCT Publication Number WO 90/02806), and the GP+envAm-12 cell line (PCT Publication Number WO 89/07150). In addition, the recombinant retroviral vectors can contain modifications within the LTRs for suppressing transcriptional activity as well as extensive encapsidation sequences which may include a part of the gag gene (Bender et al., J. Virol., 1987, 61:1639). Recombinant retroviral vectors are purified by standard techniques known to those having ordinary skill in the art.

Retroviral vectors can be constructed to function as infectious particles or to undergo a single round of transfection. In the former case, the virus is modified to retain all of its genes except for those responsible for oncogenic transformation properties, and to express the heterologous gene. Non-infectious viral vectors are manipulated to destroy the viral packaging signal, but retain the structural genes required to package the co-introduced virus engineered to contain the heterologous gene and the packaging signals. Thus, the viral particles that are produced are not capable of producing additional virus.

Retrovirus vectors can also be introduced by DNA viruses, which permits one cycle of retroviral replication and amplifies transfection efficiency (See, PCT Publication Numbers WO 95/22617, WO 95/26411, WO 96/39036 and WO 97/19182.).

In another embodiment, lentiviral vectors can be used as agents for the direct delivery and sustained expression of a transgene in several tissue types, including brain, retina, muscle, liver, and blood. The vectors can efficiently transduce dividing and nondividing cells in these tissues, and maintain long-term expression of the gene of interest. For a review, see, Naldini, Curr. Opin. Biotechnol., 1998, 9:457-63; see also, Zufferey et al., J. Virol., 1998, 72:9873-80. Lentiviral packaging cell lines are available and known generally in the art. They facilitate the production of high-titer lentivirus vectors for gene therapy. An example is a tetracycline-inducible VSV-G pseudotyped lentivirus packaging cell line that can generate virus particles at titers greater than 106 IU/ml for at least 3 to 4 days (Kafri et al., J. Virol., 1999,73: 576-584). The vector produced by the inducible cell line can be concentrated as needed for efficiently transducing non-dividing cells in vitro.

Other molecules are also useful for facilitating transfection of a nucleic acid in vitro, such as a cationic oligopeptide (e.g., PCT Patent Publication Number WO 95/21931), peptides derived from DNA binding proteins (e.g., PCT Patent Publication Number WO 96/25508), or a cationic polymer (e.g., PCT Patent Publication Number WO 95/21931), or bupivacaine (U.S. Pat. No. 5,593,972).

The isolated polypeptide of the present invention can be delivered to the mammal using a live vector, in particular using live recombinant bacteria, viruses, or other live agents, containing the genetic material necessary for the expression of the polypeptide or immunogenic fragment as a foreign polypeptide. Particularly, bacteria that colonize the gastrointestinal tract, such as Salmonella, Shigella, Yersinia, Vibrio, Escherichia and BCG have been developed as vaccine vectors, and these and other examples are discussed by Holmgren et al. (1992) and McGhee et al. (1992).

The following might be used as part of a list of vectors, without limitation:

Classification of Nonsegmented, Negative-Sense, Single Stranded RNA Viruses of the Order Mononegavirales

Family Paramyxoviridae

Subfamily Paramyxovirinae

Genus Paramyxovirus
Sendai virus (mouse parainfluenza virus type 1)
Human parainfluenza virus (PIV) types 1 and 3
Bovine parainfluenza virus (BPV) type 3

Genus Rubulavirus
Simian virus 5 (SV) (Canine parainfluenza virus type 2)
Mumps virus
Newcastle disease virus (NDV) (avian Paramyxovirus 1)
Human parainfluenza virus (PIV-types 2, 4a and 4b)

Genus Morbillivirus
Measles virus (MV)
Dolphin Morbillivirus
Canine distemper virus (CDV)
Peste-des-petits-ruminants virus
Phocine distemper virus
Rinderpest virus

Unclassified
Hendra virus
Nipah virus
   Subfamily Pneumovirinae

Genus Pneumovirus
Human respiratory syncytial virus (RSV)
Bovine respiratory syncytial virus
Pneumonia virus of mice

Genus Metapneumovirus
Human metapneumovirus
Avian pneumovirus (formerly Turkey rhinotracheitis virus)
   Family Rhabdoviridae

Genus Lyssavirus
Rabies virus

Genus Vesiculovirus
Vesicular stomatitis virus (VSV)

Genus Ephemerovirus
Bovine ephemeral fever virus
   Family Filovirdae

Genus Filovirus
Marburg virus

The RNA virus vector is basically an isolated nucleic acid molecule that comprises a sequence which encodes at least one genome or antigenome of a nonsegmented, negative-sense, single stranded RNA virus of the Order Mononegavirales. The isolated nucleic acid molecule may comprise a polynucleotide sequence which encodes a genome, antigenome, or a modified version thereof. In one embodiment, the polynucleotide encodes an operably linked promoter, the desired genome or antigenome, and a transcriptional terminator.

In a preferred embodiment of this invention, the polynucleotide encodes a genome or antigenome that has been modified from a wild-type RNA virus by a nucleotide insertion, rearrangement, deletion, or substitution. The genome or antigenome sequence can be derived from a human or non-human virus. The polynucleotide sequence may also encode a chimeric genome formed from recombinantly joining a genome or antigenome from two or more sources. For example, one or more genes from the A group of RSV are inserted in place of the corresponding genes of the B group of RSV; or one or more genes from bovine PIV (BPIV), PIV-1 or PIV-2 are inserted in the place of the corresponding genes of PIV-3; or RSV may replace genes of PIV and so forth. In additional embodiments, the polynucleotide encodes a genome or anti-genome for an RNA virus of the Order Mononegavirales which is a human, bovine, or murine virus. Since the recombinant viruses formed by the methods of this invention are employed for therapeutic or prophylactic purposes, the polynucleotide may also encode an attenuated or an infectious form of the RNA virus selected. In many embodiments, the polynucleotide encodes an attenuated, infectious form of the RNA virus. In particularly preferred embodiments, the polynucleotide encodes a genome or antigenome of a nonsegmented, negative-sense, single stranded RNA virus of the Order Mononegavirales having at least one attenuating mutation in the 3′ genomic promoter region and having at least one attenuating mutation in the RNA polymerase gene, as described by published International patent application WO 98/13501, which is hereby incorporated by reference.

As vectors, the polynucleotide sequences encoding the modified forms of the desired genome and antigenome as described above also encode one or more genes or nucleotide sequences for the immunogenic proteins of this invention. In addition, one or more heterologous genes may also be included in forming a desired immunogenic composition/vector, as desired. Depending on the application of the desired recombinant virus, the heterologous gene may encode a co-factor, cytokine (such an interleukin), a T-helper epitope, a restriction marker, adjuvant, or a protein of a different microbial pathogen (e.g., virus, bacterium, or fungus), especially proteins capable of eliciting a protective immune response. The heterologous gene may also be used to provide agents which are used for gene therapy. In preferred embodiments, the heterologous genes encode cytokines, such as interleukin-12, which are selected to improve the prophylactic or therapeutic characteristics of the recombinant virus.

Preferably, the vector is an adenovirus. More preferably, the adenovirus is a replication-defective adenovirus. Even more preferably, the replication-defective adenovirus comprises an SV40 promoter, a CMV promoter, an MLP promoter or combinations thereof. Even more preferably, the replication-defective adenovirus comprises an SV40 promoter.

High Throughput Screening

The methods for detecting and identifying a SHP inhibitor in a biological sample in accordance with the present invention contemplate the use of High Throughput Screens. There are three different phases in the flow of information from a high-throughput screen: experimental design for biochemical assays, data integrity issues and data analysis. Experimental design is most important when setting up a screen and is used to quickly identify optimal assay conditions and ensure the quality of the resulting data. Data integrity issues involve calculation of assay variability for different sets of compounds, identification and possibly correction of systematic effects and verification that the data is appropriate for its intended use. Finally, data analysis involves operations with the data, either data reduction to summarize raw data in terms of a result like Ki, or extraction of patterns and information from data, such as an SAR, QSAR or neural network analysis. Taken together, these methods address the challenge of extracting information from volumes of data.

Experimental design is a classical statistical method that has found utility in many disciplines that require finding optimal conditions and modeling response surfaces but has often been underutilized in biology. The theoretical basis for experimental design is described in detail in numerous statistical texts, e.g., Cochran, W. G. and Cox, G. M., Experimental Designs, John Wiley and Sons: New York, 1957; Hicks, C. R., Fundamental Concepts of Design of Experiments, Holt, Rhinehart and Winston, 1974; Box, G. E., Hunter, W. G. and Hunter, S., Statistics for Experimenters: An Introduction to Design, Data Analysis and Model Building, John Wiley and Sons: New York, 1978; Montgomery, D. C., Design and Analysis of Experiments, John Wiley and Sons: New York, 1984, each of which is incorporated herein by reference in its entirety. Application case studies can be found in the literature associated with a given field. Examples include industrial process optimization as described in Daniel, C., Applications of Statistics to Industrial Experimentation, John Wiley and Sons: New York, 1976; Murphy, T. D., design and analysis of industrial experiments, Chemical Engineering, Jun. 6, 1977, 168-182; and chemistry as described in Deming, S. N. and Morgan, S. L., Experimental Design: a chemometric approach; Elsevier, 1987; Austel, V., Design of Test Series by Combined Apllication of 2n Factorial Schemes and Pattern Recognition Techniques. Quantitative Approaches to Drug Discovery; Dearden, J. C., Ed.; Elsevier, 1983. Problems and methodologies associated with using experimental design for biological and chemical applications are disclosed by Haaland, Experimental Design in Biotechnology, Marcel Dekker, 1989.

Consideration is given to means for optimizing in a systematic way in order to extract the most information in the fewest numbers of runs. In many cases, identification of the optimal conditions is sufficient. In other cases, the relationship between the factors and the response is modeled mathematically. Interactions are particularly important since they are more the rule than the exception in biology and can greatly increase the complexity of a problem or in worst case, make interpretation of the data impossible. A special subset of experimental designs are variance reduction experimental designs. In such designs, the variance of a response is optimized rather than the response itself. These designs are important in biology since by minimizing variance the assay becomes more robust. In other applications, cost is an important factor and optimization is desired to minimize use of scarce resources like proteins. In many cases multiple responses are to be considered: for example, without limitation, minimizing variance while keeping receptor concentration low in order to conserve protein. Finally, the advantage to having a model, even a simple one such as the linear model that forms the basis of classical experimental designs, is that you can make predictions.

In classical experimental design, investigators choose a number of factors that they believe may have an effect on the response. Depending on the number of factors and their possible ranges, different types of designs are chosen to sample the space defined by these factors. All of the factors are varied simultaneously in a systematic way that later allows measuring the significance of a specific factor or interaction between factors. Typically the factors are sampled at the extremes of the operating range and a linear or polynomial interpolating function is used to model the region between the design points.

Different experimental designs are chosen for specific objectives. When a robotic assay is first set up, there are often many factors that potentially can influence the robustness of the assay. To find the important factors, a low resolution fractional factorial design might be run. After important factors are identified, a full factorial design might be set up to find the optimal settings for the factors. Response surface modeling could be done to explore the region around the optimal settings, to assess sensitivity of the factors and to test for higher order interactions.

Recent scientific and technological advances have introduced new paradigms for drug discovery research. The availability of chemical libraries and robotic systems for bioassay allow synthesis and testing of hundreds or even thousands of compounds in a single day. This provides for assay automation and data analysis. Combinatorial libraries provide a large number of compounds for testing. The vast amount of data that becomes available when libraries are tested against an array of molecular targets creates new opportunities for structure-activity relationship analysis and amplifies the need for effective statistical methods to ensure the integrity of the data and identify trends and relationships in the data.

The present invention provides a novel, large scale screen for agents that inhibit expression of a luciferase reporter gene that is expressed in specific tissue or cell types. The approach combines a agent library with a high throughput in situ procedure and subsequent analysis. A large number of compounds may be tested in one run by use of robotic technology. For example, about 1,000 to 40,000 compounds may be tested in one day. Preferably, about 40,000 compounds are tested in one day.

In accordance with an embodiment of the present invention, the screen utilizes frozen vials of cells which are robotically aliquoted into well plates. The well plates are then incubated and the plates are removed and assayed for the reporter. Preferably, 40 million cells are used per batch, and 1000 cells are aliquoted into each well of 100 384 well plates (a well plate having 384 well). Preferably, the incubation is conducted at about 37° C. for about 24 to about 48 hours.

Agents

The foregoing methods of the present invention are able to identify agents effective in inhibiting SHP and/or FXR in the inflammatory gene pathway and/or cholesterol biosynthesis pathway. The agents that may be identified by the methods of the present invention include small molecules, antisense oligonucleotides, recombinant receptors (e.g., recombinant SHP, recombinant FXR, soluble recombinant SHP, soluble recombinant FXR) antibodies and the like, without limitation.

In accordance with an embodiment of the invention, the present invention provides small molecules that are effective in inhibiting SHP or FXR activity in the inflammatory gene expression pathway and/or cholesterol biosynthesis pathway, such as a small molecule characterized as causing an increase in luciferase when administered to a cell culture infected with a vector comprising a NF-κB promoter/luciferase (luc) gene reporter, said cell culture expressing SHP or FXR.

According to an implementation of the invention, the small molecules are characterized as causing an increase in luciferase when administered to a cell culture infected with a vector comprising a CYP7A1 or CYP8B1 promoter/luciferase (luc) gene reporter, said cell culture expressing short heterodimer protein (SHP) and optionally hepatocyte nuclear factor 4α (HNF4α).

The term “small molecule” is used for its ordinary meaning and common usage in the art to which the invention pertains, as would be readily understood by persons of ordinary skill in the art. Preferably, the small molecule has a molecular weight of about 50 to about 1500. More preferably, the molecular weight of the agent is about 50 to about 750. Even more preferably, the molecular weight of the small molecule is about 50 to about 500. The molecular weight is determined according to any accepted method for measuring the molecular weight of small molecules as would be known to persons skilled in the art.

The small molecule may be of a wide variety of classes of compounds. For example, without limitation, the small molecule may be a natural or synthetic lipid. Further, the small molecule may range from fat-soluble to water soluble. According to an implementation of the present invention, the small molecule is fat soluble.

According to an implementation of the present invention, the small molecule is a natural or synthetic steroid. Steroids are a large group of naturally occurring and synthetic lipids or fat-soluble chemicals that exhibit a great diversity of physiological activity. Included among the steroids are certain alcohols (sterols), bile acids, many important hormones, some natural drugs, and the poisons found in the skin of some toads. Various sterols found in the skin of human beings are transformed into vitamin D when they are exposed to the ultraviolet rays of the sun. In fact, cholesterol itself is a sterol.

Steroid hormones, which are similar to but not identical with sterols, include the adrenal cortical hydrocortisone, cortisone, aldosterone, and progesterone; and the female and male sex hormones estrogen and testosterone. Most oral contraceptives are synthetic steroids consisting of female sex hormones that inhibit ovulation. Cortisone and various synthetic derivatives of cortisone are widely used in medicine. For example, such steroids are used for a variety of skin ailments, rheumatoid arthritis, asthma and allergies, and various eye diseases, and in cases of adrenal insufficiency, or the malfunctioning of the adrenal cortex. Not every small molecule of the invention is a steroid and not every steroid is a small molecule of the invention. Based upon the guidance provided herein, persons of ordinary skill in the art would readily know how to identify a particular small molecule of the invention.

According to a further implementation of the present invention, the small molecule is a nonsteroidal compound. Based upon the guidance provided herein, persons of ordinary skill in the art would readily know how to identify a particular small molecule according to this implementation of the present invention.

According to an implementation of the present invention, the small molecule is preferably a nonsteroidal compound. More preferably, the small molecule is a nonsteroidal compound having a molecular weight of about 50 to about 1500. Even more preferably, the small molecule is a nonsteroidal compound having a molecular weight of about 50 to about 750. Even more preferably, the small molecule is a nonsteroidal compound having a molecular weight of about 50 to about 500.

According to an implementation of the present invention, the small molecule is preferably a non-estrogen steroid hormone. More preferably, the small molecule is a non-estrogen steroid hormone having a molecular weight of about 50 to about 1500. Even more preferably, the small molecule is a non-estrogen steroid hormone having a molecular weight of about 50 to about 750. Even more preferably, the agent is a non-estrogen steroid hormone having a molecular weight of about 50 to about 500.

According to an implementation of the present invention, the small molecule binds to the mature or immature form of SHP or FXR or gene encoding same. Further, an implementation of the present invention contemplates a small molecule that competes with the mature or immature form of SHP or FXR for a receptor or ligand, said small molecule being present in the composition in an amount effective against atherosclerosis. Additionally, the present invention contemplates small molecules that are SHP or FXR antagonists. An antagonist is a structural analog that binds to a receptor without triggering the normal effect of the natural ligand, thereby blocking the effect of the natural ligand. Accordingly, a SHP or FXR antagonist would compete at a ligand and/or receptor binding site to prevent SHP or FXR from binding, thus inhibiting the activity of SHP or FXR. Based upon the guidance provided herein, a person of ordinary skill in the art would readily be able to identify SHP or FXR antagonists and prepare same in accordance with embodiments of the present invention.

Therapeutic Compositions

Also provided are therapeutic compositions. The compositions of the invention are useful for treating a broad range of conditions, as described above. For example, the therapeutic compositions of the present invention can be used for the treatment of elevated serum low density lipoprotein (LDL) cholesterol levels in mammals, such as humans (preferably) and non-human animals. For example, the animals may be bovine, canine, equine, feline, and porcine. Particular applications include, but are not limited to, the treatment of atherosclerosis and other conditions related to elevated LDL cholesterol levels.

The therapeutic compositions of the invention may either be prophylactic (i.e., to prevent infection or reduce the onset of infection) or therapeutic (i.e., to treat a disease or side effects caused by an infection after the infection).

The compositions may comprise a agent of the invention. To do so, one or more types of agents are adjusted to an appropriate concentration and can be formulated with any suitable diluent, carrier, or any combination thereof. Physiologically acceptable media may be used as carriers and/or diluents. These include, but are not limited to, water, an appropriate isotonic medium, glycerol, ethanol and other conventional solvents, phosphate buffered saline, and the like.

Once formulated, the compositions of the invention can be administered directly to the subject, delivered ex vivo to cells derived from the subject, or in vitro. For delivery directly to the subject, administration may be by any conventional form, such as intranasally, parenterally, orally, intraperitoneally, intravenously, subcutaneously, or topically applied to any mucosal surface such as intranasal, oral, eye, lung, vaginal, or rectal surface, such as by an aerosol spray.

Any biologically-acceptable dosage form, and combinations thereof, are contemplated by the inventive subject matter. Examples of such dosage forms include, without limitation, chewable tablets, quick dissolve tablets, effervescent tablets, reconstitutable powders, elixirs, liquids, solutions, suspensions, emulsions, tablets, multi-layer tablets, bi-layer tablets, capsules, soft gelatin capsules, lard gelatin capsules, caplets, lozenges, chewable lozenges, beads, powders, granules, particles, microparticles, dispersible granules, cachets, douches, suppositories, creams, topicals, inhalants, aerosol inhalants, patches, particle inhalants, implants, depot implants, ingestibles, injectables, infusions, health bars, confections, animal feeds, cereals, cereal coatings, foods, nutritive foods, functional foods and combinations thereof. The preparation of the above dosage forms is well known to persons of ordinary skill in the art. The compositions of the present invention are preferably in an oral dosage form. These dosage forms are all well-known to persons of ordinary skill in the art.

The term “pharmaceutically-acceptable”, as used herein, means that the dosage form must be of sufficiently high purity and suitable for use in contact with cells, tissues, or membranes without undue toxicity, incompatibility, instability, allergic response, and the like.

In a preferred embodiment, the therapeutic composition of the subject invention is administered orally to a biological subject. Also, the therapeutic composition of the invention is administered in the form of a soup. It is believed that any flavoring or food may be added to the soup to alter taste as desired.

Various additives may be incorporated into the present composition. Optional additives of the present composition include, without limitation, starches, sugars, fats, antioxidants, amino acids, proteins, derivatives thereof or combinations thereof.

It is also possible in the pharmaceutical composition of the inventive subject matter for the dosage form to combine various forms of release, which include without limitation, immediate release, extended release, pulse release, variable release, controlled release, timed release, sustained release, delayed release, long acting, and combinations thereof. The ability to obtain immediate release, extended release, pulse release, variable release, controlled release, timed release, sustained release, delayed release, long acting characteristics and combinations thereof is performed using well known procedures and techniques available to the ordinary artisan. Each of these specific techniques or procedures for obtaining the release characteristics is well known to those of ordinary skill in the art. As used herein, a “controlled release form” means any form having at least one component formulated for controlled release. As used herein, “immediate release form” means any form having at least some of its pharmaceutically active components formulated for immediate release.

The following procedures represent, without limitation, acceptable methods of preparing formulations failing within the scope of the inventive subject matter.

Quick dissolve tablets may be prepared, for example, without limitation, by mixing the formulation with agents such as sugars and cellulose derivatives, which promote dissolution or disintegration of the resultant tablet after oral administration, usually within 30 seconds.

Cereal coatings may be prepared, for example, without limitation, by passing the cereal formulation, after it has been formed into pellets, flakes, or other geometric shapes, under a precision spray coating device to deposit a film of active ingredients, plus excipients onto the surface of the formed elements. The units thus treated are then dried to form a cereal coating.

Health bars may be prepared, without limitation, by mixing the formulation plus excipients (e.g., binders, fillers, flavors, colors, etc.) to a plastic mass consistency. The mass is then either extended or molded to form “candy bar” shapes that are then dried or allowed to solidify to form the final product.

Soft gel or soft gelatin capsules may be prepared, for example, without limitation, by dispersing the formulation in an appropriate vehicle (vegetable oils are commonly used) to form a high viscosity mixture. This mixture is then encapsulated with a gelatin based film using technology and machinery known to those in the soft gel industry. The industrial units so formed are then dried to constant weight.

Chewable tablets, for example, without limitation, may be prepared by mixing the formulations with excipients designed to form a relatively soft, flavored, tablet dosage form that is intended to be chewed rather than swallowed. Conventional tablet machinery and procedures, that is both direct compression and granulation, or slugging, before compression, can be utilized. Those individuals involved in pharmaceutical solid dosage form production are well versed in the processes and the machinery used as the chewable dosage form is a very common dosage form in the pharmaceutical industry.

Film coated tablets, for example, without limitation, may be prepared by coating tablets using techniques such as rotating pan coating methods or air suspension methods to deposit a contiguous film layer on a tablet. This procedure is often done to improve the aesthetic appearance of tablets, but may also be done to improve the swallowing of tablets, or to mask an unpleasant odor or taste, or to improve properties of an unsightly uncoated tablet.

Compressed tablets, for example, without limitation, may be prepared by mixing the formulation with excipients intended to add binding qualities to disintegration qualities. The mixture is either directly compressed or granulated, then compressed using methods and machinery quite well known to those in the industry. The resultant compressed tablet dosage units are then packaged according to market need, i.e., unit dose, rolls, bulk bottles, blister packs, etc.

For example, animal feed may be made by methods well known to persons of ordinary skill in the art. Animal feeds may be prepared by mixing the formulation with binding ingredients to form a plastic mass. The mass is then extruded under high pressure to form tubular (or “spaghetti-like”) structures that are cut to pellet size and dried.

The present inventive subject matter contemplates pharmaceutical compositions formulated for administration by any route, including without limitation, oral, buccal, sublingual, rectal, parenteral, topical, inhalational, injectable and transdermal, preferably oral. The physicochemical properties of nutritional compositions, their formulations, and the routes of administration are important in absorption. Absorption refers to the process of nutritional composition movement from the site of administration toward the systemic circulation. Orally administered nutritional compositions maybe in the form of tablets or capsules primarily for convenience, economy, stability, and patient acceptance. They must disintegrate and dissolve before absorption can occur. Using the present inventive subject matter, with any of the above routes of administration or dosage forms, is performed using well known procedures and techniques available to the ordinary skilled artisan.

The present inventive subject matter contemplates the use of biologically-acceptable carriers which may be prepared from a wide range of materials. Without being limited thereto, such materials include diluents, solvents, binders and adhesives, lubricants, plasticizers, disintegrates, colorants, bulking substances, flavorings, sweeteners and miscellaneous materials, such as buffers and adsorbents in order to prepare a particular medicated composition.

Binders may be selected from a wide range of materials, such as hydroxypropylmethylcellulose, ethylcellulose, or other suitable cellulose derivatives, povidone, acrylic arid methacrylic acid co-polymers, pharmaceutical glaze, gums, milk derivatives, such as whey, starches, and derivatives, as well as other conventional binders well known to persons skilled in the art. Exemplary non-limiting non-toxic solvents are water, ethanol, isopropyl alcohol, methylene chloride or mixtures and combinations thereof. Exemplary non-limiting bulking substances include sugar, lactose, gelatin, starch, and silicon dioxide.

The plasticizers used in the dissolution modifying system are preferably previously dissolved in an organic solvent and added in solution form. Preferred plasticizers may be selected from the group consisting of diethyl phthalate, diethyl sebacate, triethyl citrate, cronotic acid, propylene glycol, butyl phthalate, dibutyl sebacate, caster oil and mixtures thereof, without limitation. As is evident, the plasticizers may be hydrophobic as well as hydrophilic in nature. Water-insoluble hydrophobic substances, such as diethyl phthalate, diethyl sebacate and caster oil are used to delay the release of water soluble materials. In contrast, hydrophilic plasticizers are used when water-insoluble materials are employed which aid in dissolving the encapsulated film, making channels in the surface, which aid in composition release.

The composition of the present inventive subject matter may be administered in a partial, i.e., fractional dose, one or more times during a 24 hour period, a single dose during a 24 hour period of time, a double dose during a 24 hour period of time, or more than a double dose during a 24 hour period of time. Fractional, double or other multiple doses may be taken simultaneously or at different times during the 24 hour period.

The compositions of the present invention are intended for use by humans and other animals. The dosages are adjusted according to body weight and thus may be set forth herein on a per body weight basis. For example, if the formula specifies a range of about 10-1000 mg for a 55 kg individual, that range would be adjusted for a 35 kg individual to about 6.3-630 mg (e.g., the lower range limit (35 kg/55 kg)*10 mg=6.3 mg). Decimal amounts may be rounded to the nearest whole number. In the above manner the present compositions may thus be adapted to be suitable for any individual, including any animal, regardless of its size.

Polypeptides

The methods of the invention screen for agents that inhibit SHP or FXR activity. In addition to SHP and FXR, the present invention contemplates utilizing any compound having SHP or FXR activity. For example, an agonist of SHP or FXR or a compound having a slight variation in sequence but the same effect as SHP or FXR. Such compounds may have usefulness in screening for inhibitors of SHP or FXR activity.

For example, a target compound may have a polypeptide sequence identical to the reference sequence of SEQ ID NOS: 2 or 4 that is, 100% identical, or it may include up to a certain integer number of amino acid alterations as compared to the reference sequence such that the % identity is less than 100%. Such alterations include at least one amino acid deletion, substitution, including conservative and non-conservative substitution, or insertion. The alterations may occur at the amino- or carboxy-terminal positions of the reference polypeptide sequence or anywhere between those terminal positions, interspersed either individually among the amino acids in the reference amino acid sequence or in one or more contiguous groups within the reference amino acid sequence.

Thus, the invention also contemplates the use of isolated polypeptides having sequence identity to the amino acid sequences contained in the Sequence Listing (i.e., SEQ ID NOS: 2 or 4). Depending on the particular sequence, the degree of sequence identity is preferably greater than 50% (e.g., 60%, 70%, 80%, 90%, 95%, 97%, 99% or more). These homologous proteins include mutants and allelic variants.

“Identity,” as known in the art, is a relationship between two or more polypeptide sequences or two or more polynucleotide sequences, as determined by comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including but not limited to those described in (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, N.J., 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48: 1073 (1988). Preferred methods to determine identity are designed to give the largest match between the sequences tested. Methods to determine identity and similarity are codified in publicly available computer programs. Preferred computer program methods to determine identity and similarity between two sequences include, but are not limited to, the GCG program package (Devereux, J., et al. 1984), BLASTP, BLASTN, and FASTA (Altschul, S. F., et al., 1990. The BLASTX program is publicly available from NCBI and other sources (BLAST Manual, Altschul, S., et al., NCBI NLM NIH Bethesda, Md. 20894; Altschul, S., et al., 1990). The well known Smith Waterman algorithm may also be used to determine identity.

For example, the number of amino acid alterations for a given % identity can be determined by multiplying the total number of amino acids in one of a plurality of sequences by the numerical percent of the respective percent identity (divided by 100) and then subtracting that product from said total number of amino acids in the plurality of sequences, or:
n_{α}≦x_{α}−(x_{α}·y),
wherein n_{α} is the number of amino acid alterations, x_{α} is the total number of amino acids in the plurality of sequences, and y is, for instance, 0.70 for 70%, 0.80 for 80%, 0.85 for 85% etc., and wherein any non-integer product of x_{α} and y is rounded down to the nearest integer prior to subtracting it from x_{α}.

Modifications and changes may be occur in the structure of SHP or FXR, but the compound may retain SHP or FXR activity. For example, certain amino acids can be substituted for other amino acids in a sequence without appreciable loss of activity and/or antigenicity. Because it is the interactive capacity and nature of a polypeptide that defines that polypeptide's biological functional activity, certain amino acid sequence substitutions can be made in a polypeptide sequence (or, of course, its underlying DNA coding sequence) and nevertheless obtain a polypeptide with like properties.

The invention thus contemplates any isolated polypeptide which is a biological equivalent that provides the substantially the same activity as would be know to a person of ordinary skill in the art reactivity as described herein.

The invention contemplates using polypeptides that are variants of the polypeptides comprising an amino acid sequence of SEQ ID NOS: 2 or 4. “Variant” as the term is used herein, includes a polypeptide that differs from a reference polypeptide, but retains essential properties. Generally, differences are limited so that the sequences of the reference polypeptide and the variant are closely similar overall and, in many regions, identical (i.e., biologically equivalent). A variant and reference polypeptide may differ in amino acid sequence by one or more substitutions, additions, or deletions in any combination. A substituted or inserted amino acid residue may or may not be one encoded by the genetic code. A variant of a polypeptide may be a naturally occurring such as an allelic variant, or it may be a variant that is not known to occur naturally. Non-naturally occurring variants of polypeptides may be made by direct synthesis or by mutagenesis techniques.

In making such changes, the hydropathic index of amino acids can be considered. The importance of the hydropathic amino acid index in conferring interactive biologic function on a polypeptide is generally understood in the art (Kyte & Doolittle, 1982). It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are listed in parentheses after each amino acid as follows: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid residue determines the secondary and tertiary structure of the resultant polypeptide, which in turn defines the interaction of the polypeptide with other molecules, such as enzymes, substrates, receptors, antibodies, antigens, and the like. It is known in the art that an amino acid can be substituted by another amino acid having a similar hydropathic index and still obtain a functionally equivalent polypeptide. In such changes, the substitution of amino acids whose hydropathic indices are within +/−2 is preferred, those which are within +/−1 are particularly preferred, and those within +/−0.5 are even more particularly preferred.

Substitution of like amino acids can also be made on the basis of hydrophilicity, particularly where the biological functional equivalent polypeptide or peptide thereby created is intended for use in immunological embodiments. U.S. Pat. No. 4,554,101, incorporated herein by reference, states that the greatest local average hydrophilicity of a polypeptide, as governed by the hydrophilicity of its adjacent amino acids, correlates with its immunogenicity and antigenicity, i.e., with a biological property of the polypeptide.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); proline (−0.5±1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). It is understood that an amino acid can be substituted for another having a similar hydrophilicity value and still obtain a biologically equivalent and in particular, an immunologically equivalent, polypeptide. In such changes, the substitution of amino acids whose hydrophilicity values are within ±2 is preferred, those which are within ±1 are particularly preferred, and those within +0.5 are even more particularly preferred.

As outlined above, amino acid substitutions are generally, therefore, based on the relative similarity of the amino acid side-chain substituents, for example, their hydrophobicity, hydrophilicity, charge, size, and the like. Exemplary substitutions which take various of the foregoing characteristics into consideration are well known to those of skill in the art and include: arginine and lysine; glutamate and aspartate; serine and threonine; glutamine and asparagine; and valine, leucine, and isoleucine. As shown in Table XIII below, suitable amino acid substitutions include the following:

TABLE I

| Original | Exemplary Residue |
|---|---|
| Residue | Substitution |
| Ala | Gly; Ser |
| Arg | Lys |
| Asn | Gln; His |
| Asp | Glu |
| Cys | Ser |
| Gln | Asn |
| Glu | Asp |
| Gly | Ala |
| His | Asn; Gln |
| Ile | Leu; Val |
| Leu | Ile; Val |
| Lys | Arg |
| Met | Met; Leu; Tyr |
| Ser | Thr |
| Thr | Ser |
| Trp | Tyr |
| Tyr | Trp; Phe |
| Val | Ile; Leu |

Biological or functional equivalents of a polypeptide can also be prepared using site-specific mutagenesis as may be used according to an implementation of the present invention. Site-specific mutagenesis is a technique useful in the preparation of second generation polypeptides, or biologically, functionally equivalent polypeptides, derived from the sequences thereof, through specific mutagenesis of the underlying DNA. As noted above, such changes can be desirable where amino acid substitutions are desirable. The technique further provides a ready ability to prepare and test sequence variants, for example, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to 25 nucleotides in length is preferred, with about 5 to 10 residues on both sides of the junction of the sequence being altered.

In general, the technique of site-specific mutagenesis is well known in the art. As will be appreciated, the technique typically employs a phage vector which can exist in both a single-stranded and double-stranded form. Typically, site-directed mutagenesis in accordance herewith is performed by first obtaining a single-stranded vector which includes within its sequence a DNA sequence which encodes all or a portion of the SHP polypeptide sequence selected. An oligonucleotide primer bearing the desired mutated sequence is prepared, for example, by well known techniques (e.g., synthetically). This primer is then annealed to the single-stranded vector, and extended by the use of enzymes, such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected which include recombinant vectors bearing the mutation. Commercially available kits provide the necessary reagents.

The polypeptides may advantageously be cleaved into fragments for use in further structural or functional analysis, or in the generation of reagents. This can be accomplished by treating purified or unpurified polypeptides with a peptidase such as endoproteinase glu-C (Boehringer, Indianapolis, Ind.). Treatment with CNBr is another method by which peptide fragments may be produced from natural SHP or FXR polypeptides. Recombinant techniques also can be used to produce specific fragments of a SHP or FXR polypeptide.

The polypeptides may be in the form of the “mature” or “immature” protein or may be a part of a larger protein such as a fusion protein or an intermediate. The polypeptides may include an additional amino acid sequence which contains, for example, secretory or leader sequences, pro-sequences, sequences which aid in purification such as multiple histidine residues, or an additional sequence for stability during recombinant production.

Fragments of the polypeptides are also contemplated by the invention. A fragment is a polypeptide having an amino acid sequence that entirely is the same as part, but not all, of the amino acid sequence. The fragment can comprise, for example, at least 7 or more (e.g., 8, 10, 12, 14, 16, 18, 20, or more) contiguous amino acids of an amino acid sequence. Fragments may be “freestanding” or comprised within a larger polypeptide of which they form a part or region, most preferably as a single, continuous region. In one embodiment, the fragments include at least one epitope of the mature polypeptide sequence.

The polypeptides used in the method for identifying inhibitors of SHP activity can be prepared in any suitable manner. Such polypeptides include naturally occurring polypeptides, recombinantly produced polypeptides, synthetically produced polypeptides, and polypeptides produced by a combination of these methods. Means for preparing such polypeptides are well understood in the art.

Polynucleotides

The invention also provides isolated polynucleotides comprising a nucleotide sequence that encodes a polypeptide of the invention, and polynucleotides closely related thereto. The polynucleotides may be used, for example, to express SHP or a reporter gene in a cell lines, according to an implementation of the invention.

For example, without limitation a polynucleotide of the present invention comprises an isolated CYP7A1 or CYP8B1 promoter. The following are two promoters used in accordance with an embodiment of the present invention.

Human CYP7A1 promoter sequence

(SEQ ID NO:5)

tctagaggatgcacttatgtagaatactctcttgaggatgttaggtgagt
aacatgttactatatgtagtaaaatatctatgattttataaaagcactga
aacatgaagcagcagaaatgtttttcccagttctctttcctctgaacttg
atcaccgtctctctggcaaagcacctaaattaattcttctttaaaagtta
acaagaccaaattataagcttgatgaataactcattcttatctttcttta
aatgattatagtttatgtatttattagctatgcccatcttaaacaggttt
atttgttctttttacacataccaaactcttaatattagctgttgtcccca
ggtccgaatgttaagtcaacatatatttgagagaacttcaacttatcaag
tattgcaggtctctgattgctttggaaccacttctgatacctgtggactt
agttcaaggccagttactaccacttttttttttctaatagaatgaacaaa
tggctaattgtttgctttgtcaaccaagctcaagttaatggatctggata
ctatgtatataaaaagcctagcttgagtctcttttcagtggcatccttcc
ctttctaatcagagattttcttcctcagagattttggcctagatttgcaa
aatgatgaccacatctttgatttgggggattgctatagcagcatgctgtt
gtctatggcttattcttggaattaggagaaggtaagta
Human CYP8B1 promoter sequence

(SEQ ID NO:6)

ccaattcgcccttggaggtaggagcagacatgacttcaacaaggtcatgc
ccccttggcaagcatctttgagaccagagaggaagacagactagggaaag
aatgaggagataagcacgggctgctgtgaggtccaggggagcaggcaaag
gtaagagaaaaggctttaggatactaactaacatatatggagcactagca
tgagccaggcactattctaagtgcttttcaggtgttatctctttttgcct
cacggacagcacctacaaggcactgtaattatccctacttcacagatgag
ggagtggagccacagtgaggttaacttacttgaccaagggggccaagtag
gaatggaggcatttgttgagtcttctaaagatgaggaaagagtggaagtg
agattttgtaagtgcttgattcatttctaccaactgaactggcaaataaa
taaaagcatgagtaaatgggggtataaatagtctgtcagctatgggggtg
ggagtgggctcaaggcaggcttagagagaaggtgcaagagctgtctgaaa
aggtcagagcaaagcatgaagctggtgagcagctgtgaccatagctggaa
gcttctctctgagctttctcctggttacctcctcctcccctacgtgacca
gtcagccaagtgttaagtccaggggaacattttgctgcttccaagtactg
tctcactagtgttatttgccataacttgcggccacagggcaaggtccagg
tgctcagacctttacatcctggactttccaaggcctcccaaagctctctg
gcacccagggaacagtgtgcgtgtcgagagagggccggg

In accordance with an embodiment of the present invention, a CYP8B1 promoter comprising the sequence from nucleotide −514 to +303 relative to the transcription initiation site of human CYP8B1. The nucleotide sequence of human CYP8B1-514 to +303 (SEQ ID NO:7) is as follows:

```
    1 ggaggtagga gcagacatga cttcaacaag gtcatgcccc cttggcaagc atctttgaga ccagagagga agacagacta
  61 gggaaagaat gaggagataa gcacgggctg ctgtgaggtc caggggagca ggcaaaggta agagaaaagg ctttaggata
121 ctaactaaca tatatggagc actagcatga gccaggcact attctaagtg cttttcaggt gttatctctt tttgcctcac
181 ggacagcacc tacaaggcac tgtaattatc cctacttcac agatgaggga gtggagccac agtgaggtta acttacttga
241 ccaagggggc caagtaggaa tggaggcatt tgttgagtct tctaaagatg aggaaagagt ggaagtgaga ttttgtaagt
301 gcttgattca tttctaccaa ctgaactggc aaataaataa aagcatgagt aaatgggggt ataaatagtc tgtcagctat
361 gggggtggga gtgggctcaa ggcaggctta gagagaaggt gcaagagctg tctgaaaagg tcagagcaaa gcatgaagct
421 ggtgagcagc tgtgaccata gctggaagct tctctctgag ctttctcctg gttacctcct cctcccctac gtgaccagtc
481 agccaagtgt taagtccagg ggaacatttt gctgcttcca agtactgtct cactagtgtt atttgccata acttgcggcc
541 acagggcaag gtccaggtgc tcagaccttt acatcctgga ctttccaagg cctcccaaag ctctctggca cccagggaac
601 agtgtgcgt gtcgag
```

Further, the polynucleotide may comprise a gene encoding a detectable substance (referred to herein as a detectable substance gene). For example, without limitation the detectable substance gene may comprise firefly luciferase gene, β-galactosidase gene, secreted alkaline phosphatase gene, renilla luciferase gene or combination thereof. Preferably, the detectable substance gene is firefly luciferase gene. According to an implementation of the invention, the nucleotide sequence comprising the CYP7A1 or CYP8B1 promoter (for example, a CYP8B1 promoter comprising the sequence from nucleotide −514 to +303 relative to the transcription initiation site of human CYP8B1) and a detectable substance gene is cloned into a vector, such as a plasmid or adenovirus, to form a construct such as a CYP8B1 promoter/detectable substance gene reporter, without limitation. The CYP8B1 promoter/detectable substance gene reporter may be used to infect or transfect a cell line so that the cell line expresses the detectable substance when the CYP8B1 promoter is activated.

The nucleotide of the present invention may comprise a promoter or gene encoding SHP and/or FXR. The promoter or genes may be cloned into a vector comprising a NF-κB promoter and the detectable substance gene.

In accordance with an embodiment of the present invention, a SHP promoter is cloned into a vector. Preferably, the SHP promoter has the following nucleic acid sequence:

Human SHP promoter sequence

(SEQ ID NO:8)

cacaagctctgagaatctcaggctctggctgtgcaattgggccagtgggt
ccagggaaacaaggactttggagtcaggcaagatctgggctttgtcttcc
tggtgggatgaccttgggcaagtcactttagcttttttagtctcataaag
taagaatctagccttaggaagaggctgccaatattagagtgggaagtgcc
tgacacataataagtgcttagagaatggcaaccatatatatacatatata
tatatatatgtatgtatgtatgtgtatatatatatacacatatacatata
aatatacatatacatatacatatacatatacatatatatttttttgagac
aggatcttgctctgttgcccaggctggagagcagtggcatgatctcagct
cactgtaacctctgcctcccaggtttcagagtgattcttttgccttagct
tctagagtagctgggactacaggcacatgccaccatgcccggctaatttt
ttgtatttttagtagagacgggattttgccatgttggccaggctggtctt
gaactcctgacctcaaatgatcccccttcctcagcctcccaaagtgctgg
gattacaggcatgagccaccgtgcccggctggcaactatcttttattata
attctgtgagttcttctcagcagacctggcctttcaggagtggtaggaat
caggctggggataaggattctgaaggaccttattcctgcagggggcccag
aactggaatcagaggaggaggcctcctagattggacagtgggcaaagtcc
tcccagccccagggtcctggctcccttccctgtagcctgcttctggctga
caacagaagcagggccccaaggttaggcaaacaagctagtgataaggcac
ttccaggttgggccttgcattcaaggcccacccagctctggggctggctt
cctggcttagcaaaagccctagtcttttgtgcacacaagagcgggcacca
atggggacacctgctgattgtgcacctggggccttggtgccctggtacag
cctgagttaatgaccttgtttatccacttgagtcatctgataaggggcag
ctgagtgagcggcaggtggccctgtgccctgcaccggccacttcattgac
tgaggtgatatcagtgccacgtggggttcccaatgccccctcccccacca
cttccccaccattcctgccaggggcaatgtcgtgtgtttttttcaatgaa
catgacttctggagtcaaggttgttgggccattccccccgttccactcac
tgggaatataaatagcacccacagcgcagaacacagagccagagagctgg
aagtgagagcagatccctaaccatgagcaccagccaaccagggg

The nucleotide of the present invention may comprise the gene encoding short heterodimer protein (SHP) and/or the gene encoding hepatocyte nuclear factor 4α (HNF4α). The genes encoding SHP and optionally HNF4α may be cloned into a vector comprising the CYP7A1 or CYP8B1 promoter and the detectable substance gene. The nucleotide may be cloned into a single vector or multiple vectors, without limitation.

“Variant” as the term is used herein, is a polynucleotide that differs from a reference polynucleotide, but retains essential properties. Changes in the nucleotide sequence of the variant may or may not alter the amino acid sequence of a polypeptide encoded by the reference polynucleotide. Nucleotide changes may result in amino acid substitutions, additions, deletions, fusions, and truncations in the polypeptide encoded by the reference sequence. A variant of a polynucleotide may be naturally occurring such as an allelic variant, or it may be a variant that is not known to occur naturally. Non-naturally occurring variants of polynucleotides may be made by mutagenesis techniques or by direct synthesis.

The invention also includes polynucleotides capable of hybridizing under reduced stringency conditions, more preferably stringent conditions, and most preferably highly stringent conditions, to polynucleotides described herein. Examples of stringency conditions are shown in the Stringency Conditions Table below: highly stringent conditions are those that are at least as stringent as, for example, conditions A-F; stringent conditions are at least as stringent as, for example, conditions G-L; and reduced stringency conditions are at least as stringent as, for example, conditions M-R.

TABLE II
STRINGENCY CONDITIONS TABLE

| Stringency | Polynucleotide | Hybrid Length | Hybridization Temperature | Wash Temperature |
|---|---|---|---|---|
| Condition | Hybrid | (bp)^{I} | and Buffer^{H} | and Buffer^{H} |
| A | DNA:DNA | >50 | 65° C.; 1xSSC -or- | 65° C.; 0.3xSSC |
| | | | 42° C.; 1xSSC, 50% formamide | |
| B | DNA:DNA | <50 | T_{B}; 1xSSC | T_{B}; 1xSSC |
| C | DNA:RNA | >50 | 67° C.; 1xSSC -or- | 67° C.; 0.3xSSC |
| | | | 45° C.; 1xSSC, 50% formamide | |
| D | DNA:RNA | <50 | T_{D}; 1xSSC | T_{D}; 1xSSC |
| E | RNA:RNA | >50 | 70° C.; 1xSSC -or- | 70° C.; 0.3xSSC |
| | | | 50° C.; 1xSSC, 50% formamide | |
| F | RNA:RNA | <50 | T_{F}; 1xSSC | T_{F}; 1xSSC |
| G | DNA:DNA | >50 | 65° C.; 4xSSC -or- | 65° C.; 1xSSC |
| | | | 42° C.; 4xSSC, 50% formamide | |
| H | DNA:DNA | <50 | T_{H}; 4xSSC | T_{H}; 4xSSC |
| I | DNA:RNA | >50 | 67° C.; 4xSSC -or- | 67° C.; 1xSSC |
| | | | 45° C.; 4xSSC, 50% formamide | |
| J | DNA:RNA | <50 | T_{J}; 4xSSC | T_{J}; 4xSSC |
| K | RNA:RNA | >50 | 70° C.; 4xSSC -or- | 67° C.; 1xSSC |
| | | | 50° C.; 4xSSC, 50% formamide | |
| L | RNA:RNA | <50 | T_{L}; 2xSSC | T_{L}; 2xSSC |
| M | DNA:DNA | >50 | 50° C.; 4xSSC -or- | 50° C.; 2xSSC |
| | | | 40° C.; 6xSSC, 50% formamide | |
| N | DNA:DNA | <50 | T_{N}; 6xSSC | T_{N}; 6xSSC |
| O | DNA:RNA | >50 | 55° C.; 4xSSC -or- | 55° C.; 2xSSC |
| | | | 42° C.; 6xSSC, 50% formamide | |
| P | DNA:RNA | <50 | T_{P}; 6xSSC | T_{P}; 6xSSC |
| Q | RNA:RNA | >50 | 60° C.; 4xSSC -or- | 60° C.; 2xSSC |
| | | | 45° C.; 6xSSC, 50% formamide | |
| R | RNA:RNA | <50 | T_{R}; 4xSSC | T_{R}; 4xSSC |

bp^{I}: The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length can be determined by aligning the sequences of the polynucleotides and identifying the region or regions of optimal sequence complementarity.
buffer^{H}: SSPE (1xSSPE is 0.15M NaCl, 10 mM NaH₂PO₄, and 1.25 mM EDTA, pH 7.4) can be substituted for SSC (1xSSC is 0.15M NaCl and 15 mM sodium citrate) in the hybridization and wash buffers; washes are performed for 15 minutes after hybridization is complete.

T_{B} through T_{R}: The hybridization temperature for hybrids anticipated to be less than 50 base pairs in length should be 5-10EC less than the melting temperature (Tₘ) of the hybrid, where Tₘ is determined according to the following equations. For hybrids less than 18 base pairs in length, Tₘ(EC)=2(# of A+T bases)+4(# of G+C bases). For hybrids between 18 and 49 base pairs in length, Tₘ(EC)=81.5+16.6(log₁₀[Na⁺])+0.41(% G+C)−(600/N), where N is the number of bases in the hybrid, and [Na⁺] is the concentration of sodium ions in the hybridization buffer ([Na⁺] for 1×SSC=0.165 M).

Additional examples of stringency conditions for polynucleotide hybridization are provided in Sambrook, J., E. F. Fritsch, and T. Maniatis, 1989, Molecular Cloning: A Laboratory Manual, Cold Spring Harbor Laboratory Press, Cold Spring Harbor, N.Y., chapters 9 and 11, and Current Protocols in Molecular Biology, 1995, F. M. Ausubel et al., eds., John Wiley & Sons, Inc., sections 2.10 and 6.3-6.4, incorporated herein by reference.

The invention also provides polynucleotides that are fully complementary to these polynucleotides and also provides antisense sequences. The antisense sequences of the invention, also referred to as antisense oligonucleotides, include both internally generated and externally administered sequences that block expression of polynucleotides encoding the polypeptides of the invention. The antisense sequences of the invention comprise, for example, about 15-20 base pairs. The antisense sequences can be designed, for example, to inhibit transcription by preventing promoter binding to an upstream nontranslated sequence or by preventing translation of a transcript encoding a polypeptide of the invention by preventing the ribosome from binding.

The polynucleotides of the invention are prepared in many ways (e.g., by chemical synthesis, from DNA libraries, from the organism itself) and can take various forms (e.g., single-stranded, double-stranded, vectors, probes, primers). The term “polynucleotide” includes DNA and RNA, and also their analogs, such as those containing modified backbones.

When the polynucleotides of the invention are used for the recombinant production of polypeptides, the polynucleotide may include the coding sequence of the mature polypeptide or a fragment thereof, by itself, the coding sequence of the mature polypeptide or fragment in reading frame with other coding sequences, such as those encoding a leader or secretory sequence, a pre-, pro-, or prepro-protein sequence, or other fusion protein portions. For example, a marker sequence which facilitates purification of the fused polypeptide can be linked to the coding sequence. The polynucleotide may also contain non-coding 5′ and 3′ sequences, such as transcribed, non-translated sequences, splicing and polyadenylation signals, ribosome binding sites, and sequences that stabilize mRNA.

Methods of Administration

The present invention contemplates methods of administering the foregoing compositions to subjects. The subjects can be mammals or birds. Preferably, the subject is a human. An effective amount of the composition in an appropriate number of doses is administered to the subject to elicit the desired response. “Effective amount”, as used herein, means the administration of that amount to a mammalian host (preferably human), either in a single dose or as part of a series of doses, sufficient to at least cause the system of the individual treated to generate a desired response, for example, in the case of atherosclerosis, a response that reduces serum level of LDL cholesterol. Protection may be conferred by a single dose of the immunogenic composition, or may require the administration of several doses, in addition to booster doses at later times to maintain protection.

The dosage amount can vary depending upon specific conditions of the individual, such as age and weight. This amount can be determined in routine trials by means known to those skilled in the art.

Various dosage forms for administering the compositions of the present invention to a patient are described above. Any suitable dosage form for administering the compositions as determined by a person of ordinary skill in the art is contemplated by the present invention.

### EXAMPLES

The following examples are illustrative and the present invention is not intended to be limited thereto.

### Example 1

### Ethynylestradiol Inhibits IL-1β Induction of Gene Expression in the Mouse Liver

An investigation was undertaken based upon the observation that incidence of cardiovascular disease is very low in women prior to menopause, yet rises dramatically following menopause. Numerous studies that have indicated hormone replacement therapy can reduce the incidence of cardiovascular disease in postmenopausal women. Although estrogen has beneficial effects on the lipid profile, lipid changes can only partially explain the reduced incidence of disease. Inflammation is a significant component of the atherosclerotic process. To investigate the ability of estrogens to inhibit inflammation in vivo, ovariectomized female C57BL/6 mice were treated with vehicle or ethynylestradiol (EE) for four days followed by a one hour treatment with IL-1β. GeneChip analysis of liver RNA revealed approximately 100 genes induced by IL-1β. Treatment with EE inhibited induction of approximately one-third of these genes. This effect was specific for gene activation, since EE did not alter basal expression levels. EE inhibition of IL-1β gene induction was absent in ERα knockout mice. EE treatment also induced expression of many genes in the liver, including prostaglandin D synthase, whose expression is known to inhibit NFκB activation. However, treatment with genistein or raloxifene maintained the inhibition of IL-1β gene induction but did not stimulate expression of prostaglandin D synthase or other genes. These results suggest that in vivo ERα can inhibit IL-1β induced gene expression by a mechanism which does not require classical ER-mediated gene induction.

Experiments were conducted to evaluate IL-1β regulation of gene expression in the mouse liver. Ovariectomized C57BL/6 mice were pretreated by subcutaneous administration of either corn oil/ethanol vehicle or 100 ug/kg/day ethynylestradiol (EE) for 5 days. On the fifth day, the mice received an intraperitoneal injection of either PBS or 20 ug/kg IL-1β in PBS. One hour later the livers were removed and polyA RNA was prepared. Global gene expression was determined by analysis using murine 11K GeneChips from Affymetrix. For each gene, the level of expression in animals receiving vehicle pretreatment plus PBS was defined as 1.0, with the expression levels in the mice receiving vehicle pretreatment±IL-1β or EE pretreatment±IL-1β defined relative to this. The results shown are the mean from two independent experiments. Seventy five genes were reproducibly induced greater than 2-fold by the IL-1β treatment. Conversely, the expression of five genes was inhibited by IL-1β. Table III below provides the results of these experiments.

TABLE III

| | | IL-1 Induction | |
|---|---|---|---|
| Gene Name | Gene ID | Vehicle | EE treated |
| JE/MCP-1 | Scya2 | 82.3 | 64.4 |
| MIP2 | Scyb2 | 60.1 | 91.1 |
| macrophage interferon inducible | Scyb10 | 50.1 | 39.3 |
| protein 10 | | | |
| gly96 | ler3 | 42.1 | 43.8 |
| ICAM-1 | lcam1 | 40.9 | 38.4 |
| PAI-1 | Serpine1 | 37.6 | 40.8 |
| serum amyloid A 3 | Saa3 | 37.4 | 36.0 |
| M-CSF | Csf1 | 32.8 | 23.9 |
| rhoB | Arhb | 30.0 | 18.5 |
| MyD118 | GADD45b | 24.3 | 12.9 |
| IL-1m | Il1m | 23.6 | 18.9 |
| molecule possessing ankyrin-repeats | aa614971 | 22.7 | 22.7 |
| induced by LPS | | | |
| Ets transcription factor ELF3 | Elf3 | 22.5 | 6.7 |
| B94, TNF-α induced protein 2 | Tnfaip2 | 17.1 | 17.7 |
| MIG | Scyb9 | 15.0 | 5.4 |
| myocyte-enriched calcineurin | Dscrl | 11.1 | 9.3 |
| interactin protein | | | |
| A20 | Tnfaip3 | 14.0 | 11.4 |
| NF-kappa-B p105 | Nfkb1 | 13.8 | 6.2 |
| suppressor of cytokine signaling 3 | Cish3 | 13.5 | 6.0 |
| IL-1B | Il1b | | |
| calgranulin B | S100a9 | 12.5 | 25.7 |
| intercrine | Scya7 | 12.4 | 11.4 |
| apoptosis inhibitor 1 | Birc2 | 12.2 | 8.9 |
| bcl-3 | Bcl3 | 11.6 | 6.2 |
| adrenomedullin | Adm | 11.3 | 10.0 |
| Gadd45 | Gadd45a | 10.9 | 3.6 |
| interferon regulatory factor 1 | Irf1 | 10.5 | 9.0 |
| calgranulin A | S100A8 | 10.4 | 10.7 |
| apoptosis inhibitor 2 | Birc3 | 9.6 | 4.3 |
| IL-15 receptor | Il15ra | 9.2 | 2.7 |
| IxBa | Nfkbia | 8.7 | 7.4 |
| H-2Ld | H-2L | 8.2 | 4.1 |
| noctumin | Ccr4 | 7.7 | 9.0 |
| PDGF-induced KC | Gro1 | 7.3 | 6.1 |
| JAB, suppressor of cytokine | Cish1 | 7.3 | 1.4 |
| signaling 1 | | | |
| E-selectin | Sele | 7.2 | 5.2 |
| inhibitor of DNA binding 3 | idb3 | 6.0 | 3.1 |
| C/EBPδ | Cebpd | 7.0 | 5.2 |
| relB | Relb | 7.0 | 7.9 |
| guanylate nucleotide binding | Gbp2 | 6.5 | 3.6 |
| protein 2 | | | |
| LPS inducible C—C chemokine | AF030185 | 6.3 | 5.6 |
| receptor related | | | |
| hsp68 | Hsp70-3 | 6.3 | 1.5 |
| CD83 | Cd83 | 6.2 | 4.0 |
| heme oxygenase 1 | Hmox1 | 5.5 | 3.0 |
| lipocalin 2 | Lcn2 | 5.5 | 2.9 |
| GTP binding protein IRG-47 | lfi47 | 5.4 | 3.2 |
| LIX | Scyb5 | 5.2 | 2.9 |
| VCAM-1 | Vcam1 | 5.2 | 12.5 |
| NF-E2 related factor 2 (NRF2) | Nfe212 | 5.2 | 7.2 |
| Ubiquitin-conjugating E2 enzyme | Ube2v2 | 5.0 | 5.3 |
| variant 2 | | | |
| c-JUN | Jun | 4.8 | 5.6 |
| MARCKS-like macrophage protein | Mlp | 4.8 | 5.1 |
| phospholipid scramblase 1 | Plscr1 | 4.4 | 5.6 |
| T-cell specific GTPase | Tgtp | 4.4 | 2.8 |
| insulin-like growth factor binding | lgfbp1 | 4.0 | 4.3 |
| protein 1 | | | |
| inducible 6-phosphofructo-2-kinase | AA163244 | 4.0 | 3.0 |
| lkB-beta | Nfkbib | 4.0 | 2.8 |
| NFkB p100 | Nfkb2 | 4.0 | 2.8 |
| Glvr-1 | Slc20a1 | 3.9 | 5.8 |
| CD14 | Cd14 | 3.5 | 6.1 |
| m-myc | Myc | 3.0 | 3.4 |
| serum amyloid A 4 | Saa4 | 2.5 | 1.9 |
| adrenergic receptor, beta 2 | Adrb2 | 2.9 | 3.1 |
| cytokine-inducible kinase Fnk | Cnk | 2.9 | 0.3 |
| junB | Junb | 2.9 | 2.6 |
| MIP-1 gamma | Scya9 | 2.9 | 1.9 |
| regulator for ribosome resistance | Rrr | 2.8 | 3.3 |
| homolog | | | |
| metalloproteinase-disintegrin | Adamts1 | 2.7 | 4.6 |
| suppressor of cytokine signaling 2 | Cish2 | 2.7 | 1.7 |
| p65 NF-kappa B | Rela | 2.5 | 2.4 |
| T-cell death associated gene | Tdag | 2.5 | 2.2 |
| calcium binding protein A6 | S100a6 | 2.4 | 2.6 |
| PIM-1 protein kinase | Pim1 | 2.4 | 2.1 |
| transglutaminase | Tgm2 | 2.2 | 8.0 |
| protein tyrosine phosphatase 4a1 | Ptp4a1 | 2.0 | 1.2 |
| TGFB inducible early growth | Tieg | 0.31 | 0.38 |
| response | | | |
| growth factor receptor bound | Grb7 | 0.27 | 0.13 |
| protein 7 | | | |
| SHP | NrGb2 | 0.23 | 0.53 |
| G0/G1 switch gene 2 | G0s2 | 0.11 | 0.05 |
| helix-loop-helix factor HES-1 | Hes1 | 0.09 | 0.25 |

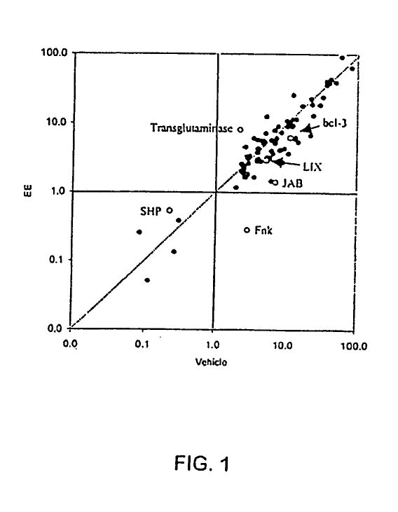

Referring to FIG. 1, the results of the study to determine whether ethynylestradiol blocks IL-1β regulation of gene expression are provided. The fold regulation of each gene is plotted for animals receiving vehicle pretreatment x-axis) vs. animals receiving pretreatment with EE (y-axis). Genes with IL-1β induction unaffected by EE pretreatment would lie along the dotted diagonal. For most genes, this is the case. However, many genes lie significantly below the diagonal line, indicating that EE inhibited induction of these genes. For example, the IL-1β inductions of bcl-3, JAB, LIX, and Fnk were all diminished by EE pretreatment (see also Table III). Expression of some genes such as transglutaminase was increased in the EE+IL-1β treated mice, consistent with the known ability of both IL-1β and estrogens to increase transglutaminase expression. Conversely, EE pretreatment inhibited the IL-1β mediated repression of SHP and HES-1, as denoted by these points lying significantly above the diagonal.

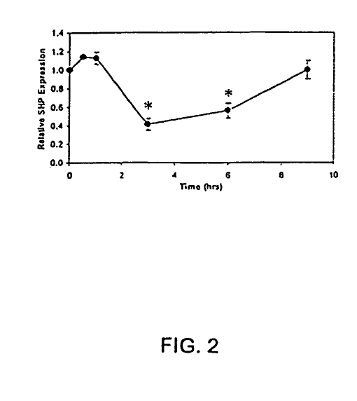

Referring to FIG. 2, IL-1β inhibition of SHP expression in HepG2 cells was examined as follows. IL-1β is typically not associated with repression of gene expression. To verify a direct IL-1β mediated repression of SHP expression, HepG2 cells were treated with 100 U/ml IL-1β. Total RNA prepared at various times afterward was analyzed for SHP expression by using realtime PCR. SHP expression was significantly reduced at 3 and 6 hours following treatment with IL-1β (*p<0.05 vs time 0). This was not due to a general decrease in cell viability as SHP mRNA levels returned to baseline levels by 9 hours following IL-1β treatment.

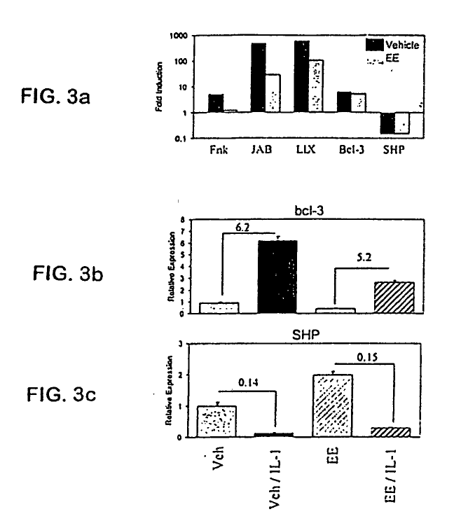

Referring to FIGS. 3a, 3b and 3c, EE Inhibition of Fnk, JAB, and LIX is Specific for IL-1β induction was studied. Two potential mechanisms could result in the apparent EE effects on gene expression. First, EE might alter the basal level of gene expression, but not influence the fold induction induced by IL-1β treatment. Alternatively, EE might have no effect on basal gene expression but rather might specifically block IL-1β mediated gene induction.

Referring to FIG. 3a, to distinguish these two mechanisms, the IL-1β fold induction of Fnk, JAB, LIX, and bcl-3 and the IL-1β fold repression of SHP was quantified in animals pretreated with vehicle (black bars) or 100 ug/kg/day EE (grey bars). Liver RNA levels of each gene (mean +/−SEM) were determined by realtime PCR of RNA from each individual animal. The fold induction (defined as the ratio of expression in animals receiving IL-1 to expression in animals receiving PBS) for Fnk, JAB, and LIX by IL-1β was reduced in EE pretreated animals. In contrast, the IL-1β fold regulation for bcl-3 and SHP was the same in vehicle and EE treated animals.

Referring to FIG. 3b, EE repressed the basal expression of bcl-3 and increased the basal expression of SHP (by comparison of EE treated mice to vehicle treated mice). EE had no effect on the direct ability of IL-1β to regulate these genes: IL-1 induction of bcl-3 was 6.2-fold in the vehicle treated mice and 5.2-fold in the EE treated mice, while IL-1 repressed SHP expression by 86% in vehicle treated animals and by 85% in EE treated animals.

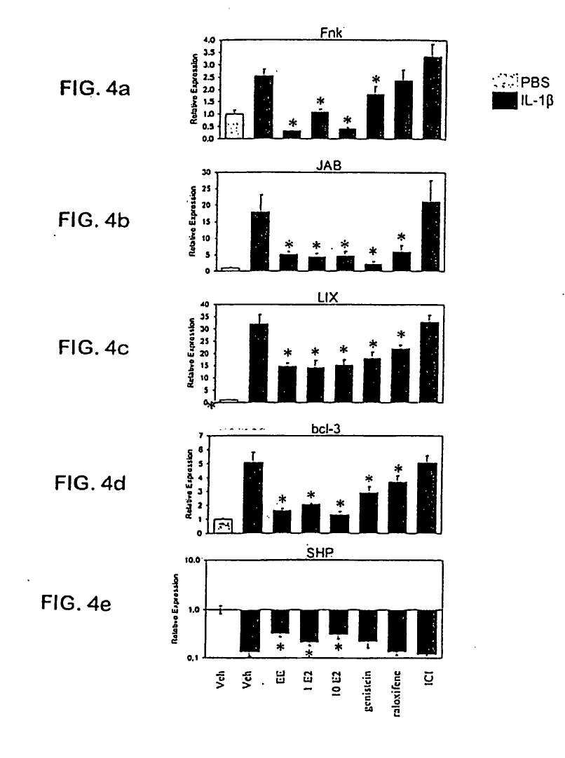

Referring to FIGS. 4a, 4b, 4c, 4d and 4e, experiments were conducted to determine whether EE blocks IL-1β induction of gene expression by an estrogen receptor (ER) dependent mechanism. Estrogens can regulate gene expression by many mechanisms including ER independent pathways such as antioxidant mechanisms. To verify that ER mediated the EE regulation of IL-1β gene regulations, mice were treated with vehicle+PBS (gray bars), vehicle+IL-1β (black bars) or 100 ug/kg/day ethynylestradiol (EE), 1 or 10 mg/kg/day 17β-estradiol (1 E2 and 10 E2, respectively), 30 mg/kg/day genistein, 5 mg/kd/day raloxifene, or 10 mg/kg/day ICI182780. Liver expression levels of Fnk, JAB, LIX, bcl-3 and SHP (mean +/−SEM) were determined by realtime PCR. EE and E2 had the greatest effect on regulation of these genes (*p<0.05 vs IL-1β alone). Genistein and the SERM raloxifene also showed as similar pattern of regulation, although the magnitude was not as great as seen with EE and E2. Finally, the pure ER antagonist ICI-182780 had no effect on expression of any of these genes. Thus the regulations of all of these genes appears to be ER mediated.

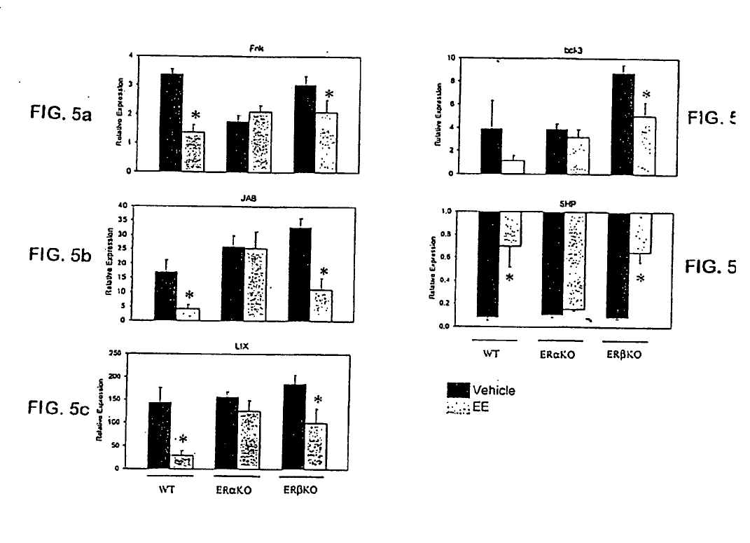

Referring to FIGS. 5a, 5b, 5c, 5d, and 5e, experiments were conducted to determine whether ERα is required for EE regulation of gene expression in the liver. To determine whether ERα or ERβ mediated the EE effects on IL-1β gene expression in the liver, C57BL/6 wild type mice, C57BL/6 ERαKO mice, or 129 ERβKO mice were pretreated with vehicle (black bars) or 10 ug/kg/day EE (gray bars) for 5 days followed by a 1 hour treatment with IL-1β. Liver expression levels of Fnk, JAB, LIX, bcl-3, and SHP (mean +/−SEM) were quantified by realtime PCR, with expression in animals receiving vehicle+PBS defined as 1.0. The pattern of EE inhibition of IL-1β regulation of all of these genes was comparable in the wild type animals and in the animals lacking ERβ (*p<0.05 for comparison of change in gene expression between vehicle and EE treated animals). In contrast, EE had no effect on IL-1β regulation of these genes in animals lacking ERα.

Referring to Table IV, EE induction of gene expression in the mouse liver was studied. One potential mechanism for EE regulation of IL-1β gene inductions was through the induction of SHP, a known transcriptional repressor. However, raloxifene was able to inhibit IL-1β induction of Fnk, JAB, LIX and bcl-3, but raloxifene did not increase SHP expression, thereby pharmacologically eliminating this mechanism (FIG. 4). To determine whether EE induction of any other gene could explain the inhibition of IL-1β gene inductions, the GeneChip results were analyzed for EE induction of gene expression. Many of the genes induced by EE pretreatment are known to be regulated by EE, including inositol-1-phosphate synthase (IPS), intestinal trefoil factor, creatine kinase, and members of the complement family. Additionally, prostaglandin D2 synthase was strongly induced by EE treatment. Overexpression of prostaglandin D synthase in vitro inhibits NFκB activation by blocking IKK activity, potentially providing an alternative mechanism for inhibition of IL-1β gene induction.

TABLE IV

| Gene Name | Gene ID | EE Induction |
|---|---|---|
| myo-inositol-1-phosphate synthase | aa221219 | 167.1 |
| prostaglandin D2 synthase | Ptgds | 72.8 |
| Keratin complex 2, basic, gene 4 | Krt2-4 | 38.0 |
| Cytochrome P450, 17 | Cyp17 | 32.3 |
| proteinase 3 | Prtn3 | 24.1 |
| cytochrome P450, 7b1 | Cyp7b1 | 21.1 |
| trefoil factor 3, intestinal | Tff3 | 16.3 |
| type llb Na/phosphate-cotransporter | Npt2b | 13.5 |
| creatine kinase B | Ckb | 13.0 |
| CD97 | Cd97 | 6.0 |
| leukemia inhibitory factor receptor | Lifr | 5.1 |
| H2A histone family, member X | H2afx | 5.1 |
| desmocollin 2 | Dsc2 | 5.0 |
| fatty acid binding protein 7, brain | Fabp7 | 4.8 |
| cell division cycle 2 homolog A | Cdc2a | 4.6 |
| serum/glucocorticoid regulated kinase | Sgk | 4.5 |
| lymphocyte antigen 86 | Ly86 | 4.3 |
| signal transducer and activator of transcription | Stat5a | 4.1 |
| 5A | | |
| cathepsin S | Ctss | 4.0 |
| lysosomal thiol reductase IP30 precursor | IP30 | 3.7 |
| solute carrier family 11, member 1 | Slc11a1 | 3.6 |
| apolipoprotein A-IV | Apoa4 | 3.5 |
| interleukin 17 receptor | Il17r | 3.5 |
| stathmin | Kist | 3.5 |
| pyrroline-5-carboxylate synthetase | Pycs | 3.3 |
| apoptosis inhibitory 6 | Api6 | 3.3 |
| F4/80 | Emr1 | 3.1 |
| CD68 antigen | Cd68 | 3.1 |
| properdin | Pfc | 2.9 |
| CD53 | Cd53 | 2.9 |
| colony stimulating factor 1 receptor | Csf1r | 2.8 |
| C1qB | C1qb | 2.8 |
| cadherin 1 | Cdh1 | 2.6 |
| mannose receptor, C type 1 | Mrc1 | 2.6 |
| interferon activated gene 203 | lfi203 | 2.5 |
| C1Qc | C1qc | 2.3 |
| testosterone 16-alpha-hydroxylase (C-16-alpha) | Cyp2d9 | 2.3 |
| apolipoprotein C2 | Apoc2 | 2.2 |

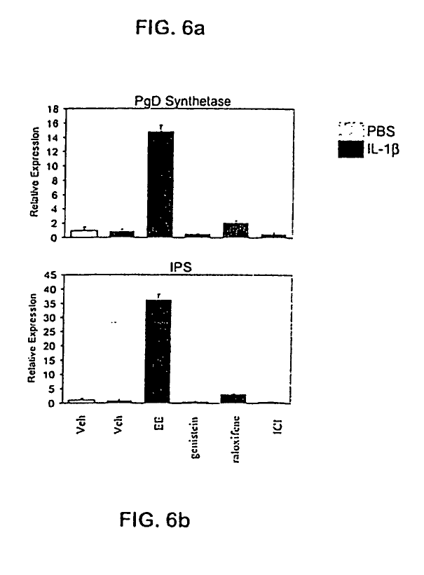

Referring to FIGS. 6a and 6b, experiments were constructed and performed to establish that EE induction of gene expression is not required for ERα inhibition of IL-1β gene inductions. To determine whether EE induction of prostaglandin D synthase was required for ERα mediated inhibition of IL-1β gene induction, the liver expression levels prostaglandin D synthase in mice treated with vehicle+PBS (gray bars), vehicle+IL-18 (black bars) or 100 ug/kg/day EE, 30 mg/kg/day genistein, 5 mg/kg/day raloxifene, or 10 mg/kg/day ICI-182780+IL-1β was determined. The expression level in the vehicle+PBS treated mice was defined as 1.0. These RNA samples were the same as used in FIG. 4, which demonstrated that these doses of EE, genistein and raloxifene all inhibited IL-1β inductions. However, there was no correlation between the ability of these compounds to inhibit IL-1β gene induction (EE>genistein>raloxifene) and the ability of these compounds to stimulate prostaglandin D synthase expression (EE>>raloxifene>genistein). A similar pattern was found for induction of IPS, with very weak induction by raloxifene and no induction by genistein. This suggests that gene induction by ERα is not required for its ability to inhibit IL-1β induction of gene expression.

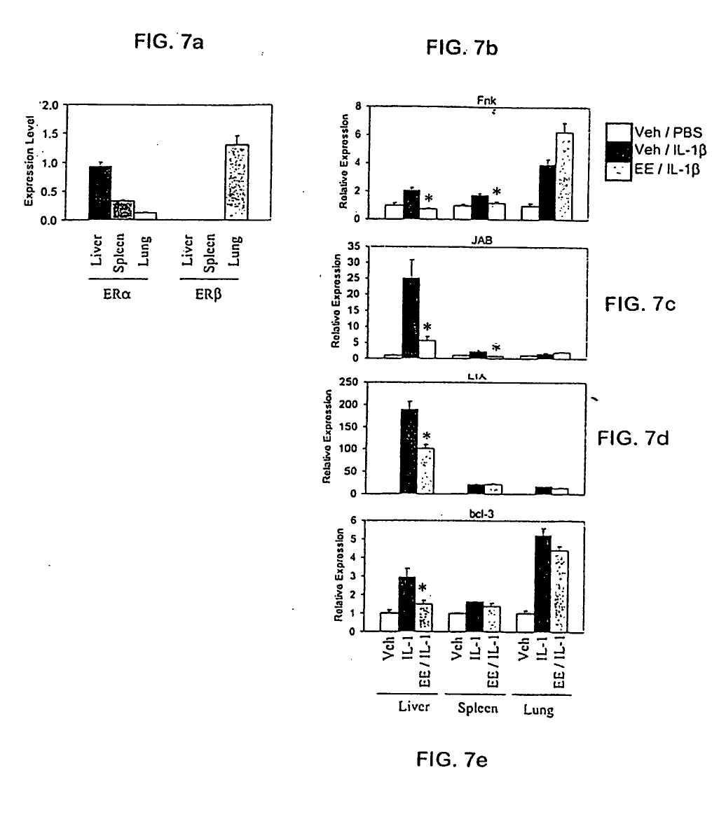

Referring to FIGS. 7a, 7b, 7c, 7d and 7e, the presence or absence of EE inhibition of IL-1β gene inductions in the lung was examined. The requirement for ERα for EE activity in the liver is consistent with the nearly exclusive presence of ERα in the liver (as determined by realtime PCR, panel A). In contrast, the lung expresses relatively low levels of ERα and high levels of ERB. To determine whether ERβ could also modulate the ability of IL-1β to induce gene expression, the ability of EE to alter IL-1β induction of Fnk, JAB, LIX, and bcl-3 was determined in the liver, spleen, and lung. As before, IL-1β induction of these genes was significantly blocked in the liver. IL-1β induction of Fnk and JAB in the spleen was also blocked by EE pretreatment. Spleen expresses significantly lower levels of ERα than liver, although somewhat higher than in the lung. In contrast, EE pretreatment had no effect on IL-1β induction of Fnk, JAB, LIX, or bcl-3 in the lung. Although tissue specific factors could influence these results, the lack of regulation in the lung agrees with the hypothesis that ERB is not able to mediate these effects and may in fact inhibit the ability of ERα to alter IL-18 induction of gene expression.

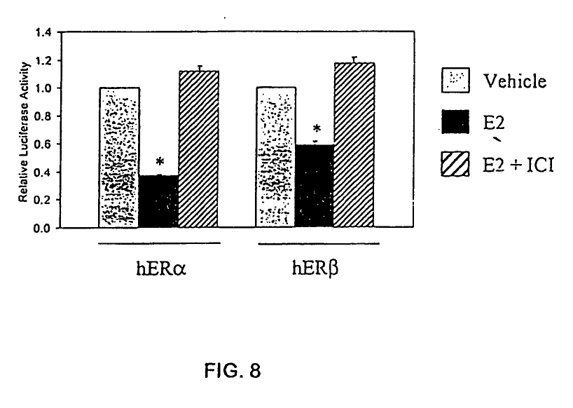

Referring to FIG. 8, ERα and ERα Inhibition of IL-1β Induction of NFκB Activity in HepG2 Cells was assessed. The lack of EE inhibition of IL-1β induction of Fnk, JAB, LIX, and bcl-3 expression in the lung could be due either to an intrinsic inability of ERβ to mediate this inhibition, or could be due to tissue specific differences between liver, spleen, and lung. To determine the ability of ERβ to directly inhibit IL-1β signaling, HepG2 cells were cotransfected with either human ERα or ERβ expression plasmids along with an NFκB driven luciferase reporter plasmid and a β-galactosidase transfection control plasmid. Following transfection the cells were treated with vehicle, 10 nM 17β-estradiol, or 10 nM 17β-estradiol+1 uM ICI182780. The following day the cells were treated with 100 U/ml IL-1β for 6 hours and assayed for luciferase expression. Both ERα and ERβ significantly repressed IL-1β induction of luciferase activity (*p<0.01), although ER13 consistently was less efficacious than ERα. Thus the lack of EE regulation of IL-1β gene induction in the lung does not appear to be due to an intrinsic inability of ERβ to inhibit IL-1β signaling. Instead, the lack of regulation in the lung may reflect tissue specific expression differences in auxiliary proteins required for ER inhibition of IL-113 signaling.

The following conclusions are evident from the results of these experiments: (1) one hour treatment with IL-1β induces expression of 75 genes and inhibits expression of 5 genes in the mouse liver; (2) EE pretreament inhibits many of these IL-11 regulations in an ERα dependent manner; (3) EE can regulate IL-1β induced gene expression both by altering basal expression levels or by specifically inhibiting gene induction; (4) ERα induction of gene expression is not necessary for the ability of ERα to block IL-1β mediated gene regulations; and (5) IL-1β Induction of Fnk, JAB, LIX, and bcl-3 is inhibited in the liver, but not in the lung, which has high levels of Erβ. This is not due to an intrinsic inability of ERβ to inhibit IL-1β signaling, but likely reflects tissue differences in cofactors necessary for EE inhibition of IL-1β signaling.

### Example 2

### Regulation of SHP Expression

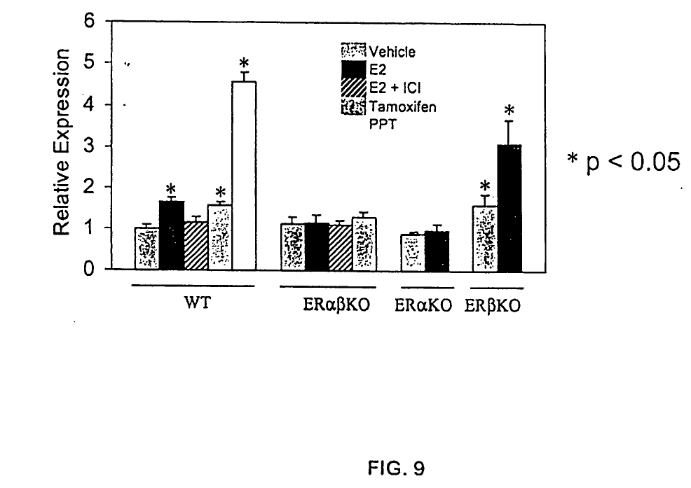

Several experiments were conducted to investigate the regulation of SHP expression, as follows. Referring to FIG. 9, ERα regulation of SHP expression in mouse liver was studied. Ovariectomized wildtype, ErαERβ double knockout, ERαKO, or ERβKO mice were treated by subcutaneous injection of vehicle, 10 ug/kg/day E2, 10 ug/kg/day E2+5 mg/kg/day ICI182780, 5 mg/kg/day tamoxifen, or 5 mg/kg/day PPT for six weeks. Liver expression of SHP was monitored by real time PCR, with normalization for GAPDH expression. In the WT animals, E2, tamoxifen, and the ERα selective agonist PPT all induced SHP mRNA levels. ICI182780 inhibited this induction. E2 did not induce the expression of SHP in either ERαKO or ERαβKO mice. In ERβKO mice the basal expression of SHP was increased, but E2 still induced expression of SHP. Together, these results indicate that estrogen induction of SHP in the mouse liver is mediated primarily by ERα.

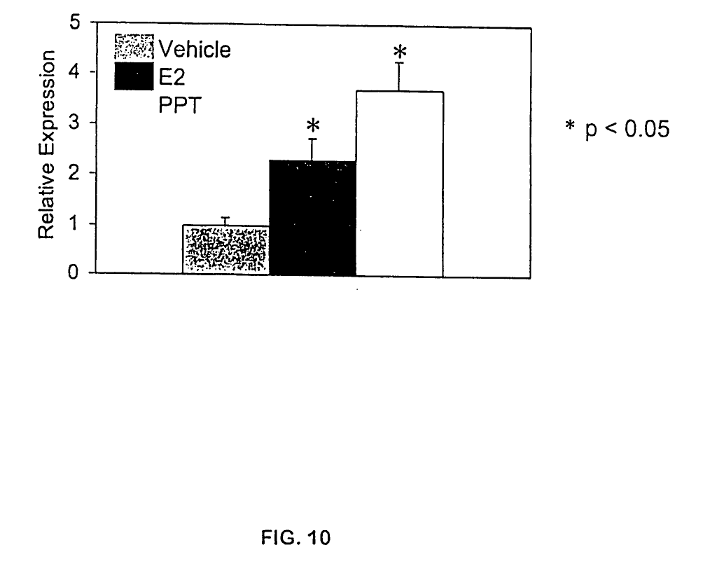

As shown in FIG. 10, a study was conducted to determine whether estrogen regulates SHP in the rat liver. Ovariectomized Sprague-Dawley rats were treated with vehicle, 10 ug/kg/day E2, or 5 mg/kg/day PPT for six weeks. Liver expression of SHP was monitored by real time PCR, with normalization for GAPDH expression. Both E2 and PPT significantly induced SHP expression, suggesting ERα also induces SHP in the rat liver.

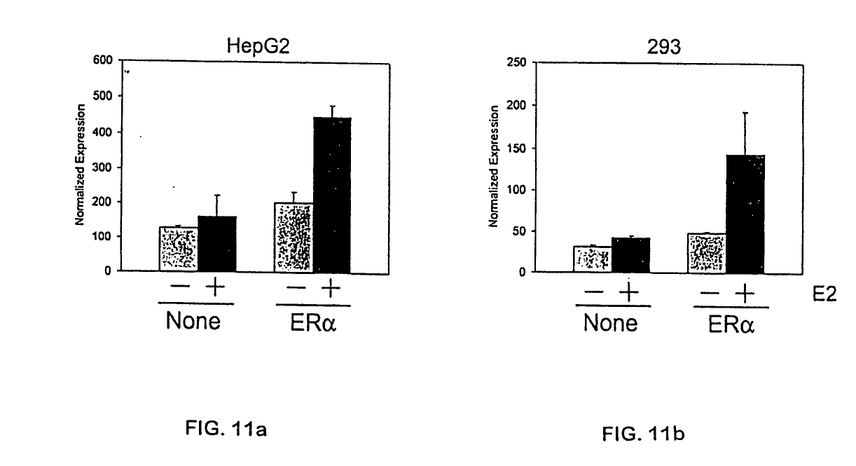

Referring to FIGS. 11a and 11b, ERα regulation of hSHP promoter activity in human cells was examined. Human HepG2 or 293 cells were cotransfected with a control SV40 driven β-galactosidase plasmid, a luceriferase plasmid driven by 1.4 kb of the human SHP promoter, and an expression plasmid encoding either no protein or human ERα. The day following transfection the cells were treated for 24 hours with DMSO vehicle or 10 nM E2. Cell extracts were then analyzed for luciferase activity normalized for β-galactosidase activity. E2 induced SHP promoter activity only in cells expressing ERα. These results suggest that E2 may regulate SHP expression in the human liver.

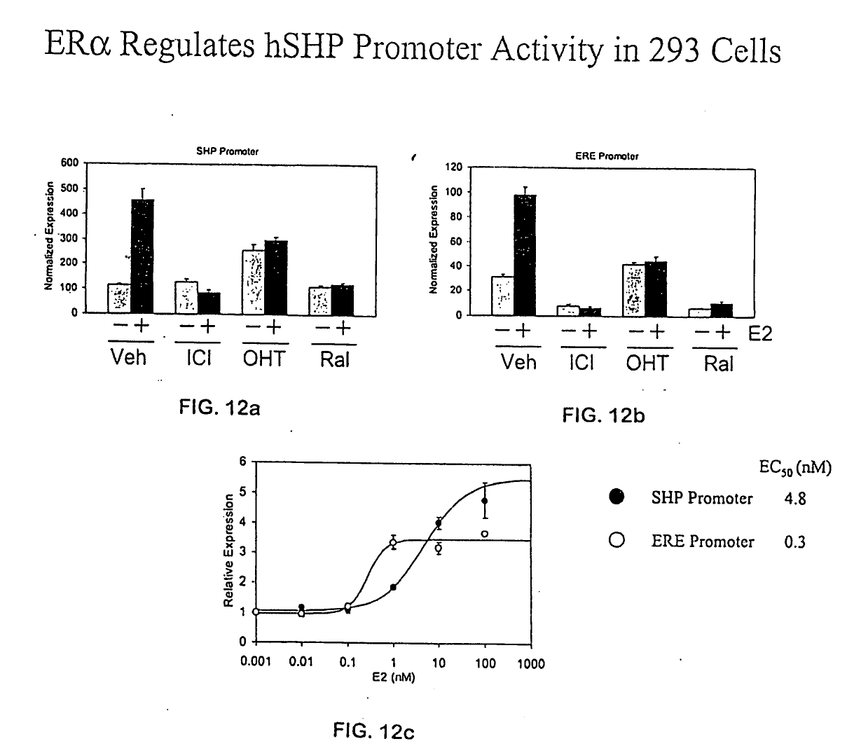

Referring to FIGS. 12a, 12b, and 12c, ERα regulation of hSHP promoter activity in 293 cells was examined. Referring to FIGS. 12a and 12b, 293 cells were cotransfected with a control SV4O driven α-galactosidase plasmid, a luceriferase plasmid driven by either 1.4 kb of the human SHP promoter or a synthetic 2×ERE/TK promoter, and an expression plasmid for human ERα. The following day the cells were treated for 24 hours with 1 uM of the indicated compounds (ICI182780, 4-hydroxytamoxifen, or raloxifene) plus DMSO vehicle (−) or 10 nM E2 (+). Cell extracts were then analyzed for luciferase activity with normalization for β-galaetosidase activity. As seen in the mouse, both E2 and tamoxifen stimulated SHP promoter activity. ICI and raloxifene were inactive alone and antagonized E2 activity. The SHP promoter differed significantly from the ERE driven promoter, suggesting that E2 regulation of SHP promoter activity may not act through a classical estrogen response element.

Referring to FIG. 12c, a dose response curve for E2 induction of SHP and 2×ERE promoter activities is provided. Approximately 10-fold higher concentrations of E2 were required for induction of SHP promoter activity than for SHP promoter activity. Since SHP is a negative regulator of ER activity, the high EC50 required for SHP promoter activation may reflect a means to only invoke this negative feedback loop when E2 levels reach high amounts.

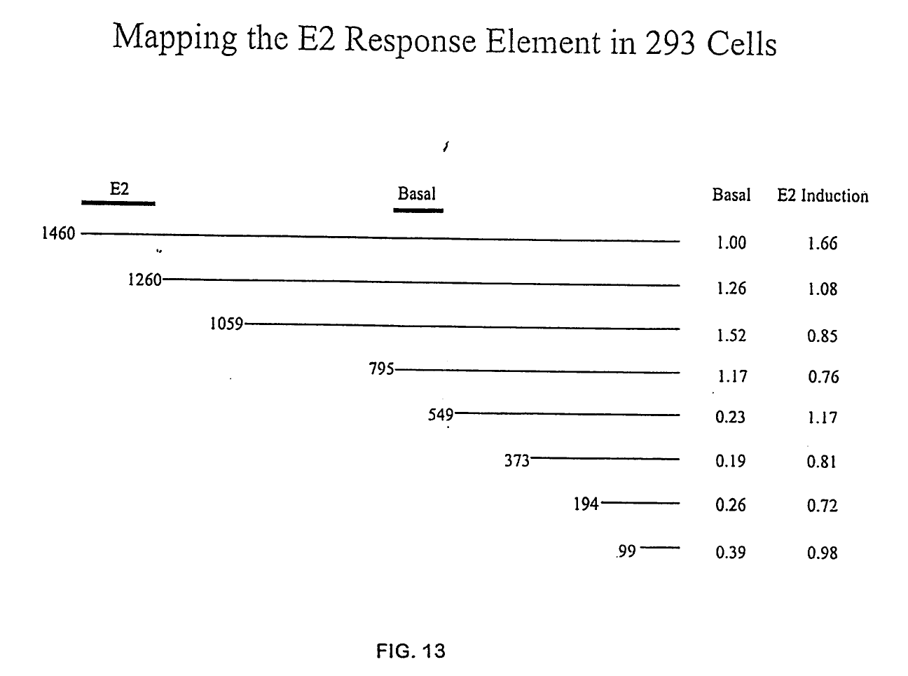

Referring to FIG. 13, a study was undertaken to map the E2 response element in 293 cells. A series of SHP promoter truncation reporter plasmids were cotransfected with an ERα expression plasmid and an β-galactosidase control plasmid into 293 cells. Normalized luciferase activity was determined after 24 hour treatment with either vehicle or 10 nM E2. The expression of the 1460 bp SHP promoter in cells treated with vehicle was defined as 1.0. An element affecting basal expression in 293 cells was localized between −795 and −549, while the E2 regulation localized between −1460 and −1260.

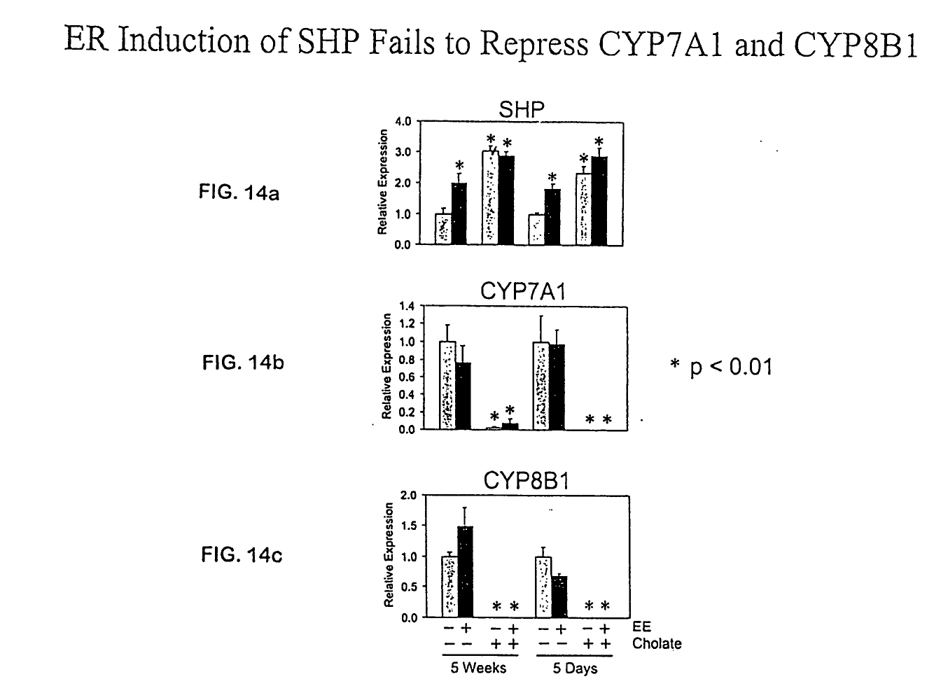

Referring to FIGS. 14a, 14b, and 14c, a study was conducted to determine whether ER induction of SHP fails to repress CYP7A1 and CYP8B1. Ovariectomized mice were fed either a control diet (−) or a diet containing cholate (+) and treated by daily subcutaneous injection of vehicle (−) or 10 ug/kg/day ethinylestradiol (+) for either 5 weeks or 5 days. At the end of the study, liver mRNA levels of SHP, CYP7A1 and CYP8B1 were determined by real time PCR with normalization for GAPDH expression. EE induced expression of SHP at either 5 days or 5 weeks of treatment to a level slightly lower than the induction seen by cholate. However, EE treatment did not significantly alter CYP7A1 or CYP8B1 expression at either time point, in contrast, cholate repressed CYP7A1 and CYP8B1 expression by greater than 95%. These results suggest that simple induction of SHP expression is not adequate for robust repression of CYP7A1 or CYP8B1.

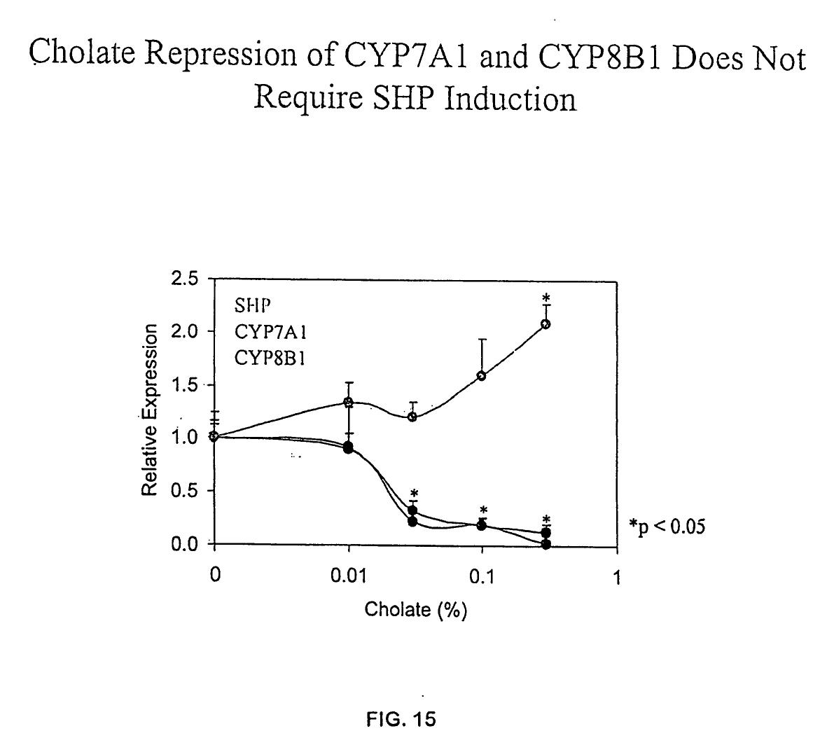

Referring to FIG. 15, experiments were conducted to examiner whether cholate repression of CYP7A1 and CYP8B1 requires SHP induction. Ovariectomized mice were fed increasing concentrations of cholate for 5 days. Liver mRNA levels for SHP, CYP7A1 and CYP8B1 were determined by real time PCR with normalization for GAPDH expression. Significant repression of CYP7A1 and CYP8B1 occurred with only 0.03% cholate in the diet, while significant elevation of SHP mRNA levels did not occur until 0.3% cholate was added to the diet. The requirement for 10-fold greater levels of cholate for SHP induction than for CYP7A1 and CYP8B1 repression again suggest that induction of SHP expression is not the sole mechanism for cholate regulation of CYP7A1 and CYP8B1 expression.

### Example 3

### Transient Transfection to Identify Compounds that Inhibit SHP in the Cholesterol Biosynthesis Pathway

A CYP8B1 promoter (the sequence from nucleotide −514 to +303 relative to the transcription initiation site) was isolated from genomic DNA by Polymerase Chain Reaction (PCR) amplification. The resulting PCR product was TOPO cloned into the plasmid pCR2.1 (available from InVitrogen, Carlsbad, Calif.) using a TOPO TA cloning kit (InVitrogen). After confirmation of the correct sequence, the CYP8B1 promoter was removed by EcoRI digestion. The ends of the resulting DNA fragment were made blunt using T4 DNA polymerase. The fragment was then ligated into Sma I digested pRL-null (available from Promega, Madison, Wis.) to create pCYP8B1-RL, having a renilla luciferase reporter driven by the human CYP8B1 promoter. The human HNF-4 and SHP coding regions were cloned by similar standard molecular methods into the SV40-promoter expression vector pSI (Promega).

The plasmids are then cotransfected in HepG2 cells. HepG2 stock cells are maintained in DMEM high glucose, 10% FBS, phenol red media (Invitrogen, GIBCO Cat. No. 11995-065). For transfection, cells are plated at 1×105 cells per well of a 12 well plate in assay medium consisting of phenol red free DMEM high glucose medium (Invitrogen, GIBCO Cat. No. 31053-028), 10% charcoal stripped FBS (HyClone, Logan, Utah, Cat. No. SH30068.03). The following day, transfection reagent Tfx-20 (Promega; E2391) is mixed with the 0.5 μg pCYP8B1-RL, 0.4 μg pSI-HNF4 and up to 1 μg pSI-SHP in serum free, phenol red free DMEM/F12 (Invitrogen, GIBCO Cat. No. 11039-021) at a 2:1 ratio of Tfx-20 to DNA, and incubated at room temperature for 10 minutes. The cells are washed once with the serum free DMEM/F12 and the medium aspirated. The Tfx-20/DNA mixture is added and the cells incubated at 37° C. for 1 hour. At the end of the incubation time, assay medium is added and the cells are incubated a further 2348 hours. The assay medium is removed and the cells are rinsed with PBS. The rinse solution is removed and 250 μl of 1× Renilla Lysis Buffer (Promega; E2810) are added. The plates are placed on a rocking platform for 15 minutes. The lysate is transferred to a microfuge tube and cleared by centrifugation for 30 seconds. A 20 μl aliquot is transferred to a microfluor plate (ThermoLab systems). 100 μl of Renilla Assay reagent is injected one well at a time and readings were taken over a 30 second interval in a Dynex MLX Microtiter Plate Luminometer.

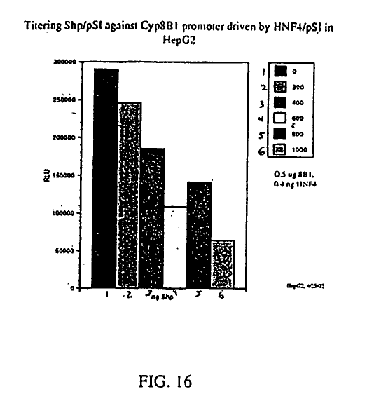

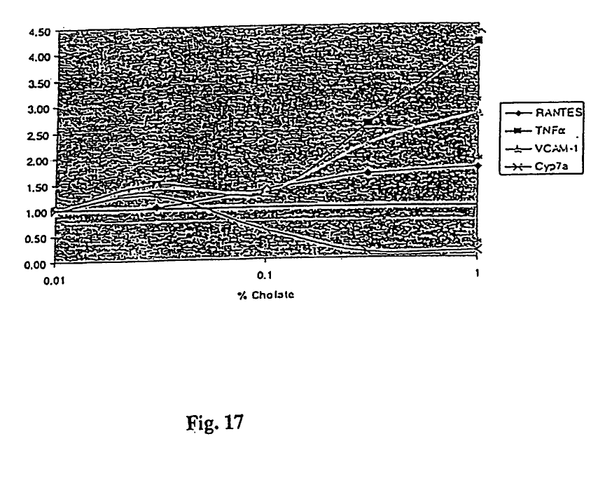

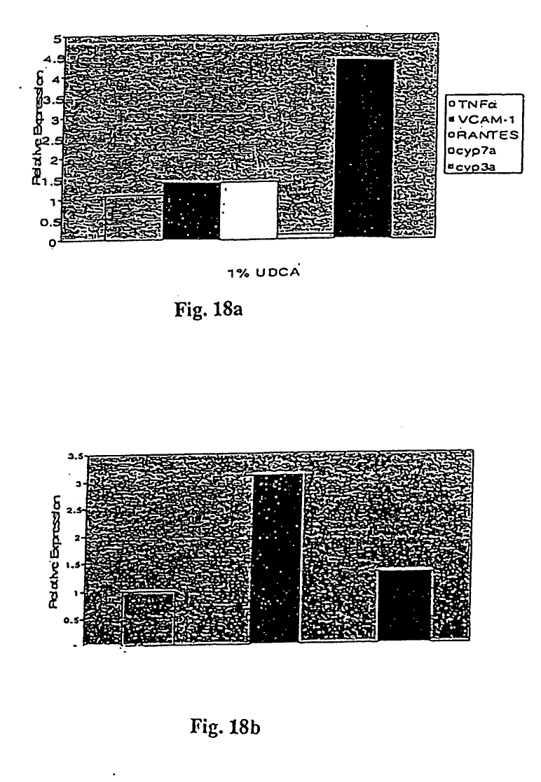

Referring to FIG. 16, the resulting luciferase activity is depicted in the graph. Addition of the SHP expression plasmid repressed expression of HNF-4 induced CYP8B1 promoter activity in a dose dependent manner.

To determine whether a test compound can inhibit SHP activity, a test compound is added to the assay medium following transfection. Luciferase activity will be increased by the presence of a SHP antagonist.

An alternative embodiment of the above method is to stably transfect cells with the pCYP8B1-RL, pSI-HNF4, and pSI-SHP plasmids using standard selectable markers including neomycin, hygromycin, or puromycin.

### Example 4

### Adenoviral Infection to Identify Compounds that Inhibit SHP in the Cholesterol Biosynthesis Pathway

A reporter adenovirus containing a region of the human CYP8B1 promoter driving expression of a reporter gene (for example firefly luciferase, renilla luciferase, or β-galactosidase) is constructed using standard techniques such as those described in the ADEASY System (available from QbioGene, Carlsbad, Calif.). The CYP8B1 promoter reporter construct is first cloned into the transfer vector pShuttle or pQBI-AdBN. The resulting plasmid is linearized with Pme I and co-transformed into E. coli strain BJ5183 together with pAdEasy-1, the viral plasmid. Recombinants are selected with kanamycin and screened by PCR or restriction enzyme analysis.

The correct plasmid is then transformed into E. coli strain DH5a and transfection quality DNA purified by typical methods such as Qiagen Maxikit. The recombinant adenoviral construct is digested with Pac I to expose its ITR (inverted terminal repeats) and transfected into QBI-293A cells to produce viral particles. The resulting plaques are used for amplification by standard methods. Following purification of the final amplified adenovirus by methods such as cesium chloride gradient, the final adenovirus stock is tittered using 293 cells. Adenoviruses expressing HNF4 and/or SHP are prepared similarly, except that alternative transfer vectors such as pShuttle-CMV may be used.

An alternative embodiment is to use any other promoter known to be responsive to HNF-4a (see Naiki et al. JBC 277:14011, 2002, incorporated by reference, for a list of such genes) to drive expression of the reporter gene.

A further alternative embodiment of the above procedure is to use a single adenovirus to express both HNF4 and SHP. In this case the transfer vector pQBI-AdCMV5-IRES-GFP is utilized. The SHP cDNA is cloned downstream of the CMV5 promoter. The GFP gene is replaced by the HNF4a gene. These steps are accomplished by standard techniques such restriction fragment recombination or more preferably by overlapping PCR. The resulting transfer plasmid contains the CMV promoter followed by the SHP cDNA followed by an internal ribosome entry segment (IRES) optionally followed by the HNF-4 cDNA. Other promoters may be used in place of the CMV promoter such as the Major Late Promoter (MLP) in the transfer plasmid pQBI-AdBM5-PAG. This preferred method ensures that SHP will be expressed in all cells in which HNF4 is expressed.

Another alternative embodiment is to use an adenovirus expressing CPF (LRH-1) in combination with a reporter adenovirus containing a region of the human CYP7A1 promoter driving expression of the reporter gene.

Once the adenoviral vectors are complete, they are used to infect human cells, most preferably human hepatoma HepG2 cells. The cells are exposed to adenovirus in medium at 37° C., typically for 5 hours. The cells are then washed and re-fed fresh medium with adenovirus. The appropriate multiplicity of infection (MOI) for the reporter plasmid and the HNF-4 expression plasmid are determined by matrix analysis using coinfection of various MOIs of each virus. Similarly, the optimal MOI for the adenovirus expressing SHP is determined. Thus in the final assay, cells are infected with the CYP8B1-reporter adenovirus at x MOI, HNF-4 expression adenovirus at y MOI, and SHP expression adenovirus at z MOI. Following infection, the cells are treated with vehicle or test compound. Test compounds is typically dissolved in DMSO and added to the culture medium in a final concentration of approximately 10 μM. 24 to 48 hours later, expression of reporter activity is measured. For example, luciferase activity may be monitored by many methods including the LUC-SCREEN Firefly Luciferase Reporter Gene Assay System (available from Applied Biosystems, Foster City, Calif.) or by lysis of the cells followed by use of a Luciferase Assay System (Promega). Compounds which increase the luciferase activity may act as SHP inhibitors.

To confirm that the test compound is acting as a SHP inhibitor, HepG2 cells are infected with the CYP8B1-reporter adenovirus at x MOI and the HNF-4 expression adenovirus at y MOI. Cells are then treated with vehicle or test compound. Luciferase activity is then monitored as described above. SHP inhibitors should not alter luciferase activity in these cells without SHP.

### Example 5-Cholate Mediates Induction of Inflammatory Gene Expression

Ovariectornized C57BL/6 mice (16-20g) (Taconic) were separated into groups of 8. After 5-7 days of recuperation, the mice were fed a casein based diet (#8117, Test Diet, Richmond, IN) for five days. The casein diet was ground to a powder and supplemented with increasing concentrations of cholic acid (CA; C-1129, Sigma, St. Louis, Mo.) or ursodeoxycholic acid (UDCA; U-5127, Sigma, St. Louis, Mo.) by mixing. At the end of the experimental period, the liver was collected for RNA analysis.

RNA Analysis

Liver total RNA was prepared using Trizol reagent (BRL) and quantitated by real time RT-PCR using an ABI PRISM 7700 Detection System according to the manufacturer's protocol (Applied Biosystem). The data was analyzed using the Sequence Dector v1.7 software (Applied Biosystems) and normalized to GAPDH using Applied Biosystems primer set.

Cell Experiments

HepG2 cells were maintained in growth media at 37° C. in a 5% CO₂ incubator. The cells were seeded in deficient growth media (phenol red free DMEM (Gibco BRL) supplemented with heat-inactivated 10% FBS, 1% Glutamax, 1% MEM non-essential amino acids, 100 U/ml penicillin and 100 μg/ml streptomycin) at 4-5×10⁵ cells per well in a 6 well dish (Falcon). The cells were then placed into serum deficient media for 24 hr before addition of the compounds for an additional 24 hrs. The cells were then harvested for RNA analysis.

Results

Feeding C57BL/6 mice a high fat diet containing cholate has been shown previously to induce inflammatory gene expression in the mouse liver after 3-5 weeks (Liao et al '93 JCI 91:2572, Evans et al. '01 Circ Res 89:823 & Miyake et al '00 JBC 275:21805). To determine the relative contribution of cholate in mediating these inductions, C57BL/6 mice were fed a chow diet supplemented with increasing concentrations of CA (0.01-1.0%) for 5 days. As shown in FIG. 1A, a dose dependent induction of the hepatic levels of TNFα (4-fold), VCAM-1 (3-fold) and RANTES (1.75-fold) mRNA was observed. As expected, a dose dependent repression of 7α-hydroxylase (cyp7a) was also observed.

These results suggested that an acute exposure to bile acids is sufficient to promote inflammatory gene expression in the absence of the oxidative stress contributed by the high fat diet in the more chronic studies or the potential toxicity associated with elevated levels of bile acids action. In vivo, CA is converted to deoxycholic acid in the intestine which has been demonstrated to selectively interact with FXR (Wang '99 Mol Cell 3:543 & Makishima '99 Science 284:1362) whereas more hydrophilic bile acids such as UDCA function through binding to pregnane X receptor (PXR) and not FXR (Heuman '89 JLR 30:1161). To determine whether signaling via FXR was necessary to result in inflammatory gene expression, 1% UDCA Bile acid signaling though binding to FXR is one of the major mechanisms of bile acid was supplemented into the chow diet. As shown in FIG. 1B, no induction of inflammatory gene expression was observed while UDCA treatment did repress cyp7a expression and induced cyp3a expression consistent with its ability to function through PXR binding.

In addition, the induction of SHP expression was also only observed with CA and not UDCA (FIG. 1C) supporting the requirement for FXR in CA signaling.

### Example 6

### FXR Specific Bile Acid Induction of SHP and RJP14O

To further investigate the possibility that bile acid signaling through FXR can promote inflammatory gene expression, experiments were conducted in the hepatocyte cell line, HepG2. HepG2 cells were treated for 24 hr with increasing concentrations of chenodeoxycholic acid (CDCA) (1-100 uM) or the selective synthetic FXR ligand, GW 4064 (1-1000 nM). CDCA was used for these experiments since CDCA is a more potent inhibitor of cyp7a expression in HepG2 cells than CA (Makishima et al '99 Science 284:1362). HepG2 cells express a number of inflammatory genes including ICAM-1 and M-CSF constitutively (Stonans et al '99 Cytokine 11:151). Both ICAM-1 and M-CSF expression was induced in a dose dependent manner by CDCA or GW 4064 treatment. As positive controls, the induction of SHP mRNA and the repression of cyp7a were confirmed. These results demonstrate that activation of FXR selectively through CDCA or GW 4064 can result in inflammatory gene induction.

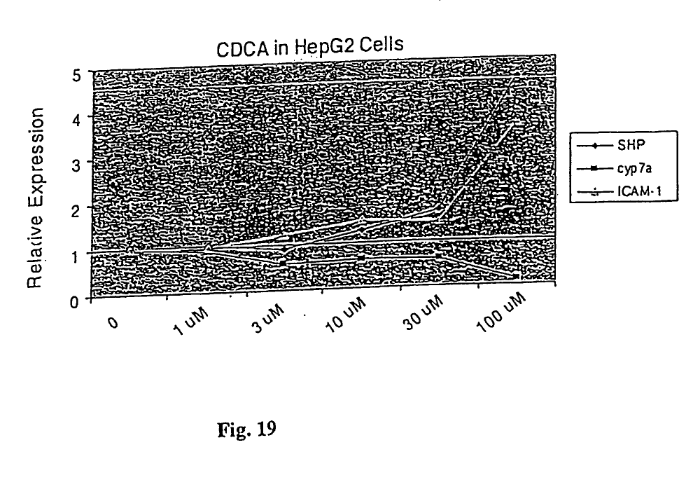

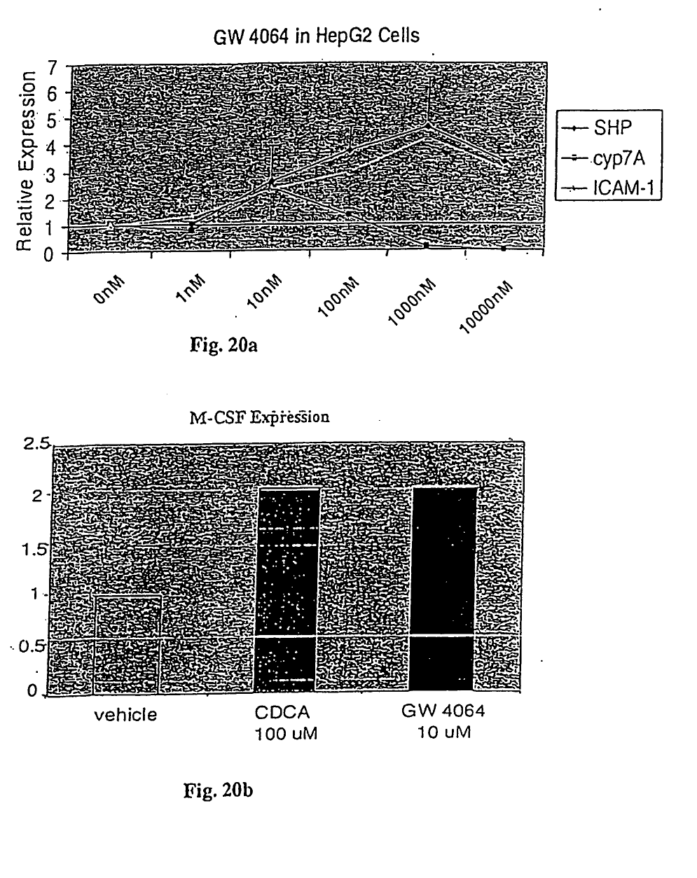

Referring to FIGS. 19, 20a and 20b relative expression of CDCA and GW 4064 in HepG2 cells is illustrated.

These experiments demonstrate a novel function of FXR in promoting inflammatory gene expression. It has previously been demonstrated that high fat diets containing bile acids can result in inflammatory gene induction in the liver of mice after 3-5 weeks (Liao et al '93 JCI 91:2572 & Evans et al. '01 Circ Res 89:823). However, these induction's were thought to occur through the oxidative stress caused by the high fat diet in combination with the bile acids and may reflect hepatic toxicity (Delzenne et al '92 Toxicity Letters 61:291). To avoid these potential confounding issues, these studies were conducted with acute bile acid exposure in chow fed mice. The results demonstrated that supplementation of 0.3% CA was sufficient to induce inflammatory gene expression. This concentration of CA also significantly inhibited cyp7a gene confirming the expected biological activity of FXR mediated gene regulation. No induction of inflammatory gene expression was observed when a more hydrophilic bile acid, UDCA, was supplemented into the chow, however repression of cyp7a was still observed. This is believed to be due to the ability of UDCA to bind to PXR (Schuetz et al '01 JBC 276:39411). Consistent with this was the induction of cyp3a activity, a well characterized PXR regulated gene. These results demonstrate that ligands for FXR and not PXR can induce inflammatory gene expression in the liver. These conclusions were supported in HepG2 cell experiments in which inflammatory gene induction of ICAM-1 and M-CSF was also observed in the presence of the bile acid CDCA or the synthetic FXR ligand, GW 4064. GW 4064 has previously been characterized to be a specific ligand for FXR (Goodwin et al '00 Mol Cell 6:5 17) with an EC₅₀ of 90 nM which is comparable to that observed for ICAM-induction as well.

Since SHP expression can be induced by bile acids or GW 4064 binding to FXR (Goodwin '00 Mol Cell 6:5 17 & Sinal '00 Cell 102:73 1), SHP mRNA levels were also monitored. We confirmed that only the FXR ligands CDCA and GW 4064 were able to induce SHP expression and not the PXR ligand UDCA. Recently, SHP and has been shown to be a coactivator for NIP-KB in vitro (Kim '01 JBC). NIP-KB is a central transcription factor involved in inflammatory gene expression and could explain how FXR ligands potentiate inflammatory gene expression.

### Example 7

### Relative Contribution of Cholate in Mediating Induction of Expression

To determine the relative contribution of cholate in mediating these inductions, C57BL/6 mice were fed a chow diet supplemented with increasing concentrations of cholic acid (CA; 0.01-1.0%) for 5 days. A dose dependent induction of the hepatic levels of TNFα, VCAM-1 and ICAM-1 mRNA was observed. A dose dependent repression of 7α-hydroxylase (cyp7a) was also observed. In vivo, CA is converted to deoxycholic acid in the intestine, which selectively interacts with FXR, whereas more hydrophilic bile acids such as ursodeoxycholic acid (UDCA) function through binding to PXR and not FXR. To determine whether signaling via FXR was necessary to result in inflammatory gene expression, 1% UDCA was supplemented into the chow diet.

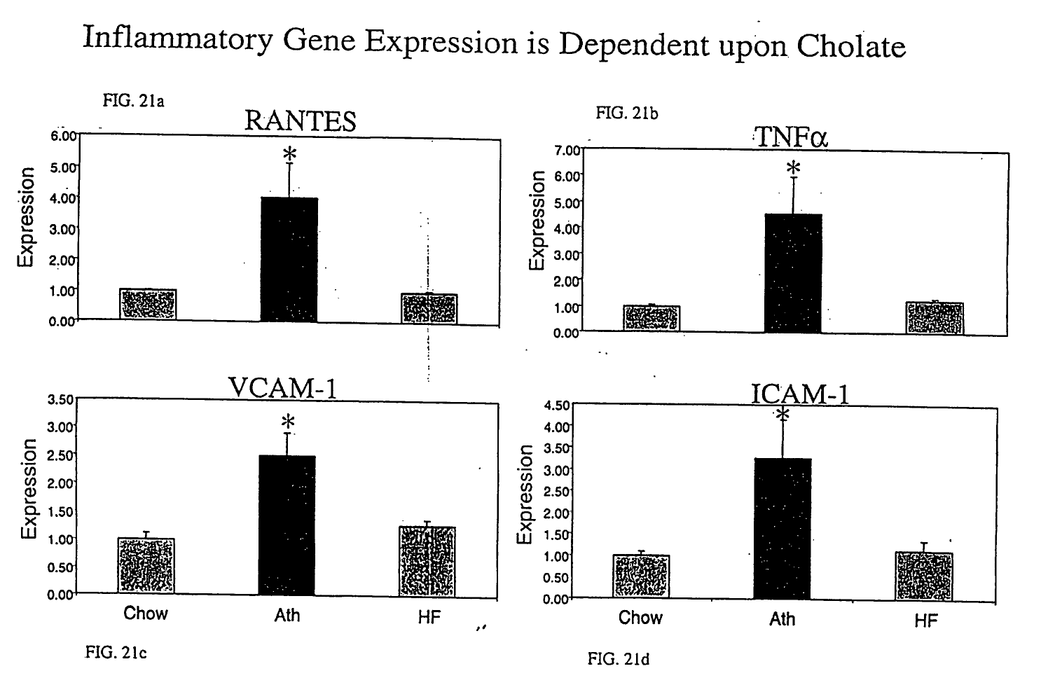

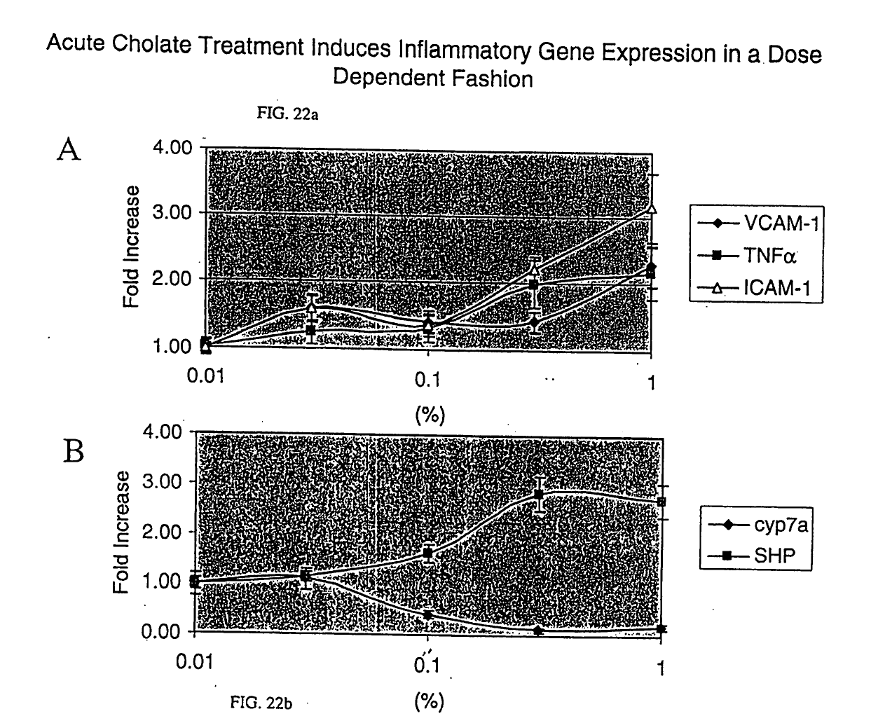

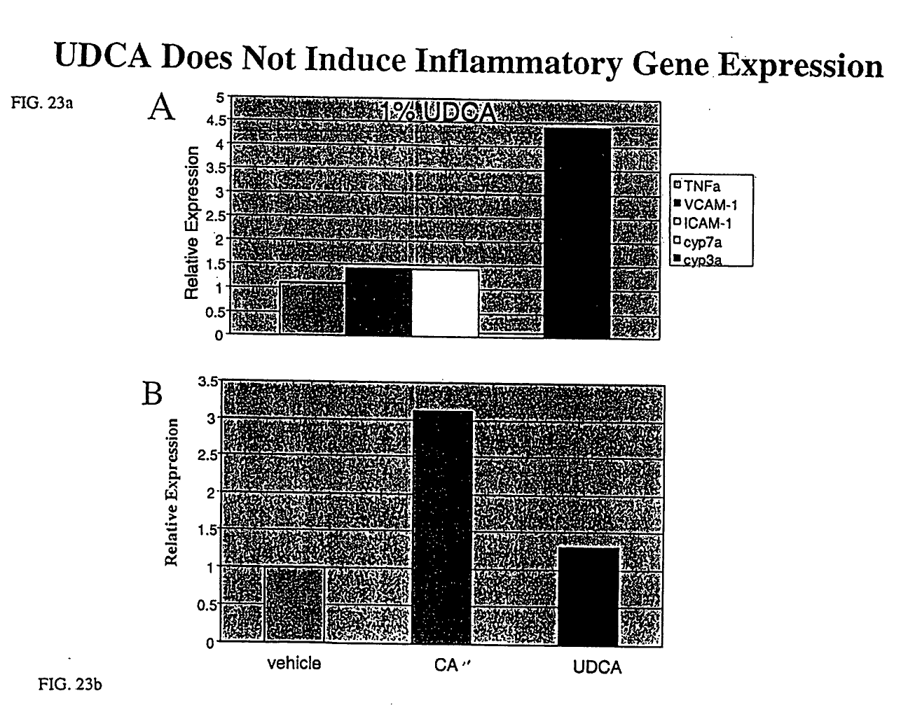

In particular, referring to FIG. 21a-d, C57BL/6 mice were fed either a chow, atherogenic or high fat diet (atherogenic diet without sodium cholate) as indicated. After 5 weeks, hepatic mRNA levels of the indicated genes were determined by real time PCR. Data are reported as mean±SEM for each group. *p<0.01 vs mice on chow diet. Referring to FIG. 22a-b, C57BL/6 mice were fed a chow diet supplemented with increasing concentrations of cholic acid as indicated. After 5 days, hepatic mRNA levels of the indicated genes were determined by real time PCR. Data are reported as mean±SEM for each group. Referring to FIG. 23a-b: C57BL/6 mice were fed a chow diet supplemented with 1% ursodeoxycholic acid (UDCA). After 5 days, hepatic mRNA levels of the indicated genes were determined by real time PCR. B. Comparison of hepatic SHP mRNA expression in mice fed either 1% cholic acid (CA) of UDCA for 5 days. Data are reported as mean±SEM for each group.

No induction of inflammatory gene expression was observed while UDCA treatment did repress cyp7a expression and induced cyp3a expression consistent with its ability to function through PXR binding. In addition, the induction of SHP expression was also only observed with CA and not UDCA treatment supporting the requirement for FXR in CA signaling.

To further investigate the possibility that bile acid signaling through FXR can promote inflammatory gene expression, experiments were conducted in the hepatocyte cell line, HepG2. HepG2 cells were treated for 24 hr with increasing concentrations of chenodeoxycholic acid (CDCA) or the selective synthetic FXR ligand, GW 4064. ICAM-1 expression was induced in a dose dependent manner by CDCA or GW 4064 treatment.

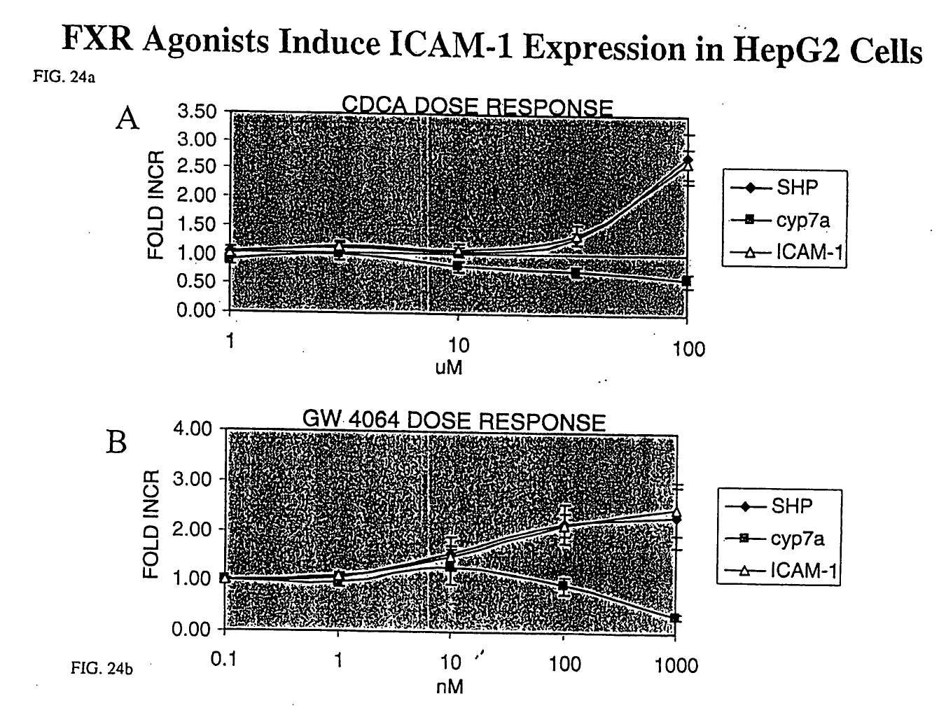

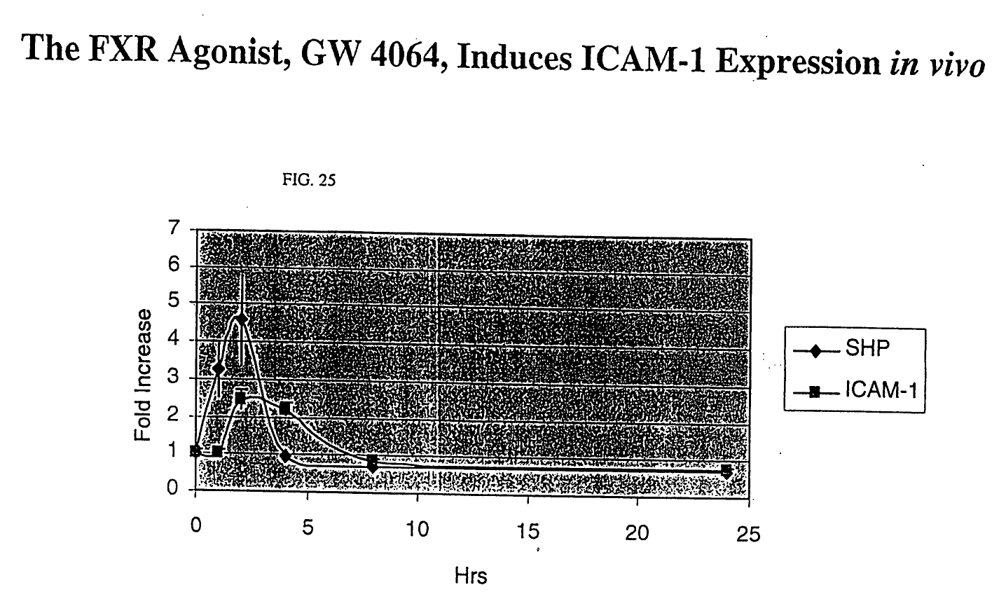

Referring to FIG. 24a-b, HepG2 cells were treated for 24 hr with increasing concentrations of chenodeoxycholic acid (CDCA). Endogenous mRNA levels of the indicated genes were determined by real time PCR. Data are reported as mean±SEM for each group B. Similar experimental design as above except cells were treated with increasing concentrations of the FXR against GW 4064. Data are reported as mean±SEM for each group. C57BL/6 mice. Referring to FIG. 25, C57BL/6 mice were treated orally with a single dose of GW 4064 (50 mg/kg). Various time points after dosing, hepatic mRNA levels of the indicated genes were determined by real time PCR. Data are reported as mean±SEM for each group.

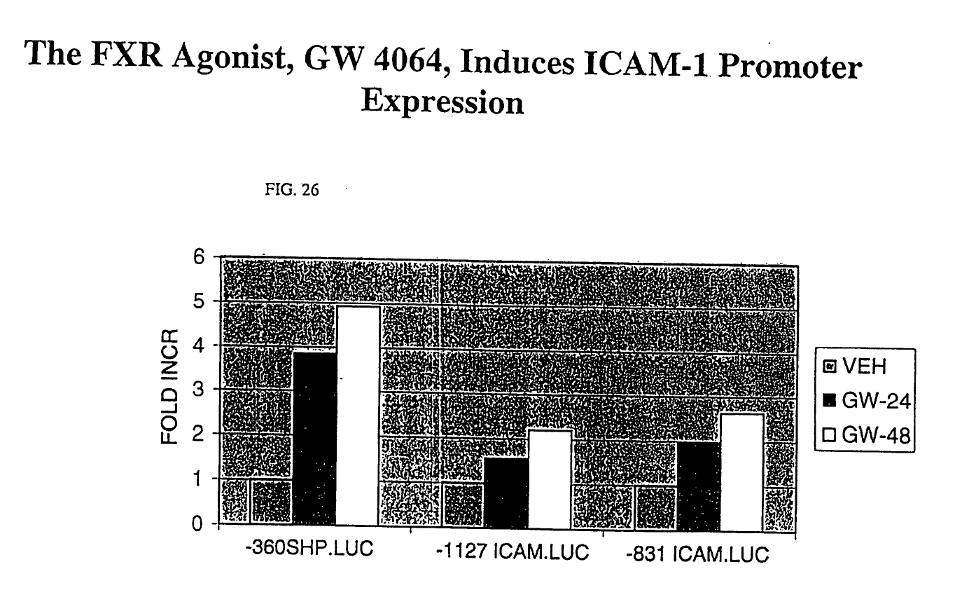

Referring to FIG. 26, HepG2 cells were transfected with the human FXR and RXRα expression plasmid and luciferase reporter plasmids containing the proximal promoter region of the human SHP (−360 to +40) or sequential deletions of the human ICAM-1 promoter (−1108 or −810 to +18). Following transfection, cells were treated for 24 or 48 hr with GW 4064 (1 uM). Data represent the mean+S.D. of three independent transfections.

As positive controls, the induction of SHP mRNA and the repression of cyp7a were confirmed for both compounds. In HepG2 cell transfections, this demonstrates that ICAM-1 promoter activity was also stimulated by GW 4064 and required the proximal NF-κB response element for its activity. SHP has been demonstrated to act as a coactivator for the p65 subunit of NF-κ, a central transcription factor involved in inflammatory gene expression. This provides evidence that FXR signaling induces NF-κB mediated inflammatory gene expression through the induction of SHP expression.

### Example 8

### Transient Transfection to Identify Compounds that Inhibit FXR and/or SHP in the Inflammatory Gene Expression Pathway

An NF-κB promoter is isolated from genomic DNA by Polymerase Chain Reaction (PCR) amplification. The resulting PCR product is TOPO cloned into the plasmid pCR2.1 (available from InVitrogen, Carlsbad, Calif.) using a TOPO TA cloning kit (InVitrogen). After confirmation of the correct sequence, the NF-κB promoter is removed by EcoRI digestion. The ends of the resulting DNA fragment are made blunt using T4 DNA polymerase. The fragment is then ligated into Sma I digested pRL-null (available from Promega, Madison, Wis.) to create pNF-κB-RL, having a renilla luciferase reporter driven by the NF-κB promoter. The human SHP or FXR coding regions is cloned by similar standard molecular methods into SV40-promoter expression vector pSI (Promega).

The plasmids are then cotransfected in HepG2 cells. HepG2 stock cells are maintained in DMEM high glucose, 10% FBS, phenol red media (Invitrogen, GIBCO Cat. No. 11995-065). For transfection, cells are plated at 1×105 cells per well of a 12 well plate in assay medium consisting of phenol red free DMEM high glucose medium (Invitrogen, GIBCO Cat. No. 31053-028), 10% charcoal stripped FBS (HyClone, Logan, Utah, Cat. No. SH30068.03). The following day, transfection reagent Tfx-20 (Promega; E2391) is mixed with the 0.5 μg pNF-κB-RL, up to 1 μg pSI-FXR or up to 1 μg pSI-SHP in serum free, phenol red free DMEM/F12 (Invitrogen, GIBCO Cat. No. 11039-021) at a 2:1 ratio of Tfx-20 to DNA, and incubated at room temperature for 10 minutes. The cells are washed once with the serum free DMEM/F12 and the medium aspirated. The Tfx-20/DNA mixture is added and the cells incubated at 37° C. for 1 hour. At the end of the incubation time, assay medium is added and the cells are incubated a further 23-48 hours. The assay medium is removed and the cells are rinsed with PBS. The rinse solution is removed and 250 μl of 1× Renilla Lysis Buffer (Promega; E2810) are added. The plates are placed on a rocking platform for 15 minutes. The lysate is transferred to a microfuge tube and cleared by centrifugation for 30 seconds. A 20 μl aliquot is transferred to a microfluor plate (ThermoLab systems). 100 μl of Renilla Assay reagent is injected one well at a time and readings were taken over a 30 second interval in a Dynex MLX Microtiter Plate Luminometer.

To determine whether a test compound can inhibit SHP activity, a test compound is added to the assay medium following transfection. Luciferase activity is increased by the presence of a SHP antagonist.

An alternative embodiment of the above method is to stably transfect cells with the pNF-κB-RL and pSI-SHP plasmids using standard selectable markers including neomycin, hygromycin, or puromycin.

### Example 9

### Adenoviral Infection to Identify Compounds that Inhibit FXR and/or SHP in the Inflammatory Gene Expression Pathway

A reporter adenovirus containing a region of the human NF-κB promoter driving expression of a reporter gene (for example firefly luciferase, renilla luciferase, or β-galactosidase) is constructed using standard techniques such as those described in the ADEASY System (available from QbioGene, Carlsbad, Calif.). The NF-κB promoter reporter construct is first cloned into the transfer vector pShuttle or pQBI-AdBN. The resulting plasmid is linearized with Pme I and co-transformed into E. coli strain BJ5183 together with pAdEasy-1, the viral plasmid. Recombinants are selected with kanamycin and screened by PCR or restriction enzyme analysis.

The correct plasmid is then transformed into E. coli strain DH5a and transfection quality DNA purified by typical methods such as Qiagen Maxikit. The recombinant adenoviral construct is digested with Pac I to expose its ITR (inverted terminal repeats) and transfected into QBI-293A cells to produce viral particles. The resulting plaques are used for amplification by standard methods. Following purification of the final amplified adenovirus by methods such as cesium chloride gradient, the final adenovirus stock is tittered using 293 cells. Adenoviruses expressing either FXR or SHP are prepared similarly, except that alternative transfer vectors such as pShuttle-CMV may be used.

An alternative embodiment is to use any other promoter known to be responsive to HNF4a (see Naiki et al. JBC 277:14011, 2002, incorporated by reference, for a list of such genes) to drive expression of the reporter gene.

A further alternative embodiment of the above procedure is to use a single adenovirus to express both HNF-4 and SHP or FXR. In this case the transfer vector pQBI-AdCMV5-IRES-GFP is utilized. The SHP cDNA is cloned downstream of the CMV5 promoter. The GFP gene is replaced by the HNF-4a gene. These steps are accomplished by standard techniques such restriction fragment recombination or more preferably by overlapping PCR. The resulting transfer plasmid contains the CMV promoter followed by the SHP cDNA followed by an internal ribosome entry segment (IRES) followed by the HNF-4 cDNA. Other promoters may be used in place of the CMV promoter such as the Major Late Promoter (MLP) in the transfer plasmid pQBI-AdBM5-PAG. This method ensures that SHP is expressed in all cells in which HNF-4 is optionally expressed.

Another alternative embodiment is to use an adenovirus expressing CPF (LRH-1) in combination with a reporter adenovirus containing a region of the human NF-κB promoter driving expression of the reporter gene.

Once the adenoviral vectors are complete, they are used to infect human cells, most preferably human hepatoma HepG2 cells. Infection is achieved by exposing the cells to adenovirus in medium at 37° C., typically for 5 hours. The cells are then washed and re-fed fresh medium with adenovirus. The appropriate multiplicity of infection (MOI) for the reporter plasmid and the SHP expression plasmid are determined by matrix analysis using coinfection of various MOIs of each virus. Thus in the final assay, cells are infected with the NF-κB-reporter adenovirus at x MOI and SHP expression adenovirus at y MOI. Following infection, the cells are treated with vehicle or test compound. Test compounds is typically dissolved in DMSO and added to the culture medium in a final concentration of approximately 10 μM. 24 to 48 hours later, expression of reporter activity is measured. For example, luciferase activity may be monitored by many methods including the LUC-SCREEN Firefly Luciferase Reporter Gene Assay System (available from Applied Biosystems, Foster City, Calif.) or by lysis of the cells followed by use of a Luciferase Assay System (Promega). Compounds which increase the luciferase activity may act as SHP inhibitors.

To confirm that the test compound is acting as a SHP or FXR inhibitor, HepG2 cells are infected with the NF-κB-reporter adenovirus at x MOI. Cells are then treated with vehicle or test compound. Luciferase activity is then monitored as described above. SHP inhibitors do not alter luciferase activity in these cells without SHP.

Although illustrated and described above with reference to specific embodiments, the invention is nevertheless not intended to be limited to the details shown. Rather, various modifications may be made in the details within the scope and range of equivalents of the claims and without departing from the spirit of the invention. Thus, the invention may be embodied in other specific forms without departing from the spirit or essential characteristics thereof. The present embodiments are, therefore, to be considered in all respects as illustrative and not restrictive, the scope of the invention being indicated by the appended claim.

1. A method for identifying an agent effective at inhibiting short heterodimer protein (SHP) or farnesoid X receptor (FXR) comprising:
administering an agent to a cell culture that expresses (i) short heterodimer protein (SHP) or (ii) farnesoid X receptor (FXR) and comprises a NF-κB promoter/detectable substance gene reporter; and
selecting agents that cause an increase in the detectable substance in the cell culture.

2. The method of claim 1, wherein the agent is a small molecule, an antisense oligonucleotide, an antibody, a recombinant SHP, a recombinant FXR or a combination thereof.

3. The method of claim 2, wherein the agent is a small molecule having a molecular weight of about 50 to about 1500.

4. The method of claim 1, wherein the detectable substance gene is firefly luciferase gene, β-galactosidase gene, secreted alkaline phosphatase gene, renilla luciferase gene or combination thereof.

5. The method of claim 1, wherein the detectable substance gene is firefly luciferase gene.

6. The method of claim 1, wherein the cell culture is an altered cell culture.

7. The method of claim 1, wherein the cell culture is a transfected cell culture.

8. The method of claim 1, wherein the cell culture is an infected cell culture.

9. The method of claim 1, wherein the SHP, FXR or promoter/detectable substance gene reporter is introduced to the cell culture by a vector selected from any of adenovirus, plasmid, retrovirus or combinations thereof.

10. The method of claim 9, wherein the vector is an adenovirus.

11. The method of claim 10, wherein the adenovirus is a replication-defective adenovirus.

12. The method of claim 11, wherein the replication-defective adenovirus comprises an SV40 promoter, a CMV promoter, an MLP promoter or a combination thereof.

13. The method of claim 12, wherein the replication-defective adenovirus comprises an SV40 promoter.

14. The method of claim 1, wherein the cell culture is any of HELA, human hepatoblastoma cell line (HepG2), human embryonic kidney 293 cell line (HEK293), rat FTO-2B, rat McA-RH7777 or combination thereof.

15. The method of claim 1, further comprising cloning NF-κB promoter for use in preparing the NF-κB promoter/detectable substance gene reporter.

16. The method of claim 1, wherein the NF-κB promoter comprises inflammatory genes intracellular adhesion molecule (ICAM-I) or macrophage-colony stimulating factor (M-CSF).

17. The method of claim 1, additionally comprising administering the agent to a second cell culture that expresses short heterodimer protein (SHP) and comprises a CYP7A1 or CYP8B1 promoter/detectable substance gene reporter to detect an increase in the detectable substance in the second cell culture.

18. The method of claim 1, additionally comprising cloning NF-κB promoter and inserting the cloned NF-κB promoter into a vector ahead of a detectable substance gene to form a NF-κB promoter/detectable substance gene reporter prior to infecting a cell culture, wherein the NF-κB promoter comprises inflammatory genes intracellular adhesion molecule (ICAM-I) or macrophage-colony stimulating factor (M-CSF).

19. The method of claim 1, additionally comprising administering the candidate agent to a second cell culture that expresses short heterodimer protein (SHP) and hepatocyte nuclear factor 4α (HNF4α) and comprises a CYP7A1 or CYP8B1 promoter/detectable substance gene reporter to detect an increase in the detectable substance in the second cell culture.

20. The method of claim 1 additionally comprising:
(a) administering the agent to a second cell culture, said second cell culture comprising a NF-κB promoter/detectable substance gene reporter and not expressing SHP or FXR; and
(b) selecting for agents that cause an increase in the detectable substance in the first cell culture and cause no increase in the detectable substance in the second cell culture following administration of the agent.

21. A method of preventing or ameliorating a condition associated with inflammatory gene activity and/or cholesterol biosynthesis in a subject comprising:
administering an agent selected by the method of claim 1 above to said subject.

22. A composition comprising an agent selected by the method of claim 1 above.

23. The composition of claim 22, which additionally comprises a pharmaceutically-acceptable carrier.

24. The composition of claim 22, wherein the agent is a small molecule, an antisense oligonucleotide, an antibody, a recombinant SHP, a recombinant FXR or a combination thereof.

25. The composition of claim 24, wherein the agent is a small molecule having a molecular weight of about 50 to about 1500.

26. A composition comprising:
an agent characterized as causing an increase in luciferase when administered to a cell culture infected, transfected or altered with a vector comprising a nuclear transcription factor NF-κB promoter/luciferase (luc) gene reporter, said cell culture expressing short heterodimer protein (SHP) or farnesoid X receptor (FXR).

27. The composition of claim 26, wherein the agent is a small molecule, an antisense oligonucleotide, an antibody, a recombinant SHP, a recombinant FXR or a combination thereof.

28. The composition of claim 27, wherein the small molecule has a molecular weight of about 50 to about 1500.

29. The composition of claim 27, wherein the small molecule has a molecular weight of about 50 to about 750.

30. The composition of claim 27, wherein the small molecule has a molecular weight of about 50 to about 500.

31. The composition of claim 27, wherein the small molecule is a nonsteroidal compound.

32. The composition of claim 26, wherein the vector is any of adenovirus, plasmid, retrovirus or combinations thereof.

33. The composition of claim 32, wherein the vector is an adenovirus.

34. The composition of claim 33, wherein the adenovirus is a replication-defective adenovirus.

35. The composition of claim 34, wherein the replication-defective adenovirus comprises an SV40 promoter, a CMV promoter, an MLP promoter or combinations thereof.

36. The composition of claim 34, wherein the replication-defective adenovirus comprises an SV40 promoter.

37. The composition of claim 26, wherein the cell culture is HELA, human hepatoblastoma cell line (HepG2), human embryonic kidney 293 cell line (HEK293), rat FTO-2B, rat McA-RH7777 or combinations thereof.

38. The composition of claim 26, wherein the NF-κB promoter comprises inflammatory genes intracellular adhesion molecule (ICAM-I) or macrophage-colony stimulating factor (M-CSF).

39. The composition of claim 26, wherein the agent inhibits the activity of short heterodimer protein (SHP) or farnesoid X receptor (FXR) in the inflammatory gene expression pathway.

40. The composition of claim 26, wherein the agent binds to the mature or immature form of short heterodimer protein (SHP) or farnesoid X receptor (FXR) or gene encoding same; said agent being present in the composition in an amount effective against atherosclerosis.

41. The composition of claim 26, wherein the agent competes with the mature or immature form of short heterodimer protein (SHP) or farnesoid X receptor (FXR) for a receptor or ligand.

42. The composition of claim 26, wherin the NF-κB promoter comprises inflammatory genes intracellular adhesion molecule (ICAM-I) or macrophage-colony stimulating factor (M-CSF).

43. A promoter/detectable substance gene reporter comprising:
an NF-κB promoter and a detectable substance gene, said NF-κB promoter positioned in front of the detectable substance gene.

44. The promoter/detectable substance gene reporter of claim 43, wherein the NF-κB promoter/detectable substance gene reporter is introduced to a vector.

45. The promoter/detectable substance gene reporter of claim 44, wherein the vector is a replication-defective adenovirus vector.

46. The promoter/detectable substance gene reporter of claim 45, wherein the replication-defective adenovirus vector comprises an SV40 promoter, a CMV promoter, an MLP promoter or combinations thereof.

47. The promoter/detectable substance gene reporter of claim 45, wherein the replication-deficient adenovirus vector comprises an SV40 promoter.

48. The promoter/detectable substance gene reporter of claim 43, wherein the composition further comprises a vector comprising a polynucleotide expressing short heterodimer protein (SHP) or farmesoid X receptor (FXR).

49. The promoter/detectable substance gene reporter of claim 43, wherein the NF-κB promoter comprises inflammatory genes intracellular adhesion molecule (ICAM-I) or macrophage-colony stimulating factor (M-CSF).

50. The promoter/detectable substance gene reporter of claim 44, wherein said vector is introduced into a host cell.

51. An isolated CYP7A1 or CYP8B1 promoter comprising:
a polynucleotide comprising the nucleic acid sequence of SEQ ID NO:5, SEQ ID NO:6, SEQ ID NO:7, SEQ ID NO:8 or a combination thereof.

52. The promoter of claim 51, wherein the CYP8B1 promoter comprises the fragment of CYP8B1 from nucleotide −514 to +303 relative to the transcription initiation site of human CYP8B1.

53. The promoter of claim 51, wherein said promoter is introduced to a detectable substance gene to form a promoter/detectable substance gene reporter.

54. The promoter of claim 53, wherein the detectable substance gene is firefly luciferase gene, β-galactosidase gene, secreted alkaline phosphatase gene, renilla luciferase gene or a combination thereof.

55. The promoter of claim 53, wherein the detectable substance gene is firefly luciferase gene.

56. The promoter of claim 53, wherein the promoter/detectable substance gene reporter is introduced into a vector.

57. The promoter of claim 56, wherein the vector is adenovirus SV40.

58. The promoter of claim 51, wherein the promoter is introduced into a host cell.

59. A composition comprising:
a non-naturally deactivated short heterodimer protein (SHP) or farnesoid X receptor (FXR) complex.

60. The composition of claim 59, which additionally comprises a pharmaceutically-acceptable carrier.

61. The composition of claim 60, wherein the pharmaceutically-acceptable carrier is a chewable tablet, quick dissolve tablet, effervescent tablet, reconstitutable powder, elixir, liquid, solution, suspension, emulsion, tablet, multi-layer tablet, bi-layer tablet, capsule, soft gelatin capsule, hard gelatin capsule, caplet, lozenge, chewable lozenge, bead, powder, granule, particle, microparticle, dispersible granule, cachet, douche, suppository, cream, topical, inhalant, aerosol inhalant, patch, particle inhalant, implant, depot implant, ingestible, injectable, infusion, health bar, confection, animal feed, cereal, cereal coating, food, nutritive food, functional food or combination thereof.

62. The composition of claim 60, wherein said composition additionally comprises a pharmaceutically-acceptable buffer, diluent, adjuvant or combination thereof.

63. A composition comprising:
an agent that binds to any of SEQ ID NOS:1-4.

64. The composition of claim 63, wherein the agent is an antisense oligonucleotide to any of SEQ ID NO:1 or SEQ ID NO:3.

65. The composition to claim 63, wherein the agent is an antibody to any of SEQ ID NO.2 or SEQ ID NO:4.

66. (canceled)

67. (canceled)
